(19) United States
(12) Patent Application Publication
(10) Pub. No.: US 2017/0073391 A1
(43) Pub. Date: Mar. 16, 2017

(54) Osteoprotegerin derived RANKL inhibitor

(71) Applicant: R-Pharm Overseas, Inc., San Diego, CA (US)

(72) Inventors: Yan Lavrovsky, Del Mar, CA (US); Ting Xu, Needham, MA (US); Irina Kostareva, Stamford, CT (US); Shorena Archuadze, Mitishchi, Moscow Region (RU); Vasily Ignatiev, Moscow (RU); Mikhail Samsonov, Moscow (RU); Alexey Repik, Moscow (RU)

(21) Appl. No.: 15/124,026

(22) PCT Filed: Mar. 6, 2014
(86) PCT No.: PCT/US2014/021417

§ 371 Date: Sep. 6, 2016

(51) Int. Cl. C07K 14/705

(52) U.S. Cl. CPC C07K 14/70578; A61K 38/00

(57) ABSTRACT

A pharmaceutical composition is described that can be used for treating or prevention of diseases association with bone resorption, particularly of a metastatic carcinoma. In certain aspects, the composition is based on a polypeptide which includes the leading 215 amino acids of the human osteoprotegerin followed by the Fc portion of the human IgG1 protein. Pharmaceutical formulations are provided that are suitable for administering the pharmaceutical composition into primates via subcutaneous and intravenous routes.

### FIELD OF THE INVENTION

Generally, the invention relates to the field of biological pharmaceuticals as well as their use in conditions associated with bone resorption, for example in oncology. More specifically, the invention relates to an osteoprotegerin-derived composition that binds to receptor activator of NF-kappaB ligand (RANKL).

### BACKGROUND

The approaches described in this section could be pursued, and are not necessarily approaches that have previously been conceived or pursued. Therefore, unless otherwise indicated, it should not be assumed that any of the approaches described in this section qualify as prior art, merely by virtue of their inclusion into this section.

Bone metastases are a common complication of both solid tumors and hematologic cancers with an incidence of 15-75% in patients with solid tumors and nearly 100% in patients with multiple myeloma. Cancers that are most likely to metastasize to bone include breast, lung, prostate, thyroid and renal cancers. Rate of bone metastases in different types of cancer is as follow:
Multiple myeloma—70-95%—Breast cancer—65-75%—Prostate cancer—65-75%—Lung cancer—30-40%—Renal cancer—40% —Bladder cancer—20-25%—Melanoma—14-45%.

Skeletal complications of bone metastases account for significant morbidity due to pain, pathologic fractures, spinal cord compression, and other nerve-compression syndromes.

Bone metastases can be osteolytic, osteoblastic, or mixed. Normal bone remodeling is controlled by osteoblasts and osteoclasts in a balanced sequence. Receptor activator of nuclear factor KB (RANK) ligand (RANKL), a member of the tumor necrosis factor family, is expressed on the surface of osteoblasts. RANKL binds the receptor RANK on osteoclast precursors, which leads to signaling via TNF receptor-associated factors (TRAFs) and ultimately activation of nuclear factor KB in the nucleus, inducing differentiation into mature osteoclasts which degrade or resorb bone. Other osteoclast-activating factors include parathyroid hormone-related protein, interleukins, and chemokines. A decoy receptor for RANKL, osteoprotegerin (OPG), is present in bone marrow and secreted by osteoblasts and acts as a balance between the osteoblasts and osteoclasts.

In the setting of bone metastases in cancer, the cross talk between RANKL, RANK, and OPG is disrupted. Osteoclast activation is enhanced when metastases release interleukins, parathyroid hormone-related protein, and other factors that up regulate RANKL expression. These factors may also inhibit OPG. In addition, growth factors released from bone lesions stimulate the growth of tumor cells, setting up a vicious cycle (Roodman G D. Mechanisms of bone metastasis. N Engl J Med 2004; 350:1655-64; Vallet S, smith M R, Rage N. Novel bone-targeted strategies in oncology. Clin Cancer Res 2010;16:4084-93; Marathe A, Peterson M C, Mager D E. Integrated cellular bone homeostasis model for denosumab pharmacodynamics in multiple myeloma patients. J Pharmacol Exp Ther 2008; 326:555-562; George S, Brenner A, Sarantopoulos J, Bukowski R M. RANK ligand: effects of inhibition. Curr Oncol Rep 2010;12: 80-86).

Human OPG (GenBank: U94332.1) is a 401 amino acid protein which contains a signal peptide of 21 amino acids, that is cleaved before glutamic acid 22 giving rise to a mature soluble protein of 380 amino acid. OPG is a member of the tumor necrosis factor receptor (TNFR) family, comprising four cysteine-rich TNFR like domains in its N-terminal portion. OPG has been shown to have a role in the development of bone, and mice lacking the OPG gene had an osteoporotic phenotype and gross skeletal abnormalities.

OPG, which is produced by osteoblasts and bone marrow stromal cells, acts as a secreted decoy receptor with no apparent direct signaling function. OPG acts by binding to its natural ligand—osteoprotegerin ligand (OPGL), which is also known as RANKL. The binding between OPG and RANKL prevents RANKL from activating its cognate receptor RANK, which is an osteoclast receptor vital for osteoclast differentiation, activation and survival.

Recombinant OPG exists in monomeric and dimeric forms of apparent molecular weights of about 55 kDa and about 110 kDa, respectively. Truncation of the N-terminal domain to residue cysteine 185 results in OPG inactivation, presumably by disrupting a disulfide bond of the TNFR-like domain, whereas truncation of the C-terminal portion of the protein to residue 194 does not alter biological activity.

Overexpression of OPG in transgenic mice leads to profound osteopetrosis characterized by a near complete lack of osteoclasts in the mice. Conversely, ablation of the OPG gene causes severe osteoporosis in mice, indicating an important physiological role of OPG in regulating bone resorption. The secretion of OPG and RANKL from osteoblasts and stromal cells is regulated by numerous hormones and cytokines. The relative levels of OPG and RANKL production are thought to control the extent of bone resorption: expression of RANKL increases bone resorption, whereas excess OPG has the opposite effect. Recombinant OPG blocks the effects of the vast majority of the factors which stimulate osteoclasts, in vitro and in vivo. OPG also inhibits bone resorption in a variety of animal disease models, including ovariectomy, induced osteoporosis, humoral hypercalcemia of malignancy, and experimental bone metastasis. Therefore, OPG might represent an effective therapeutic option for diseases associated with excessive osteoclast activity (Kostenuik P J, Shalhoub V., Curr Pharm Des. 2001 May; 7(8):613-35).

RANK/RANKL pathway is well-known target that has proved to be the effective treatment for bone metastasis. Denosumab is a high affinity monoclonal antibody that binds to human RANKL and inhibits its interactions with RANK, thus having a similar to OPG mode of action. Denosumab is a full-length human monoclonal anti-RANKL antibody of the IgG2 subclass, consisting of 2 heavy chains, and 2 light chains of the kappa subclass, produced in Chinese hamster ovary (CHO) cells. Denosumab under the trade name Prolia was approved by U.S. Food and Drug Administration (FDA) for prevention and treatment of osteoporosis in postmenopausal women. Denosumab under the trade name Xgeva was approved by U.S. Food and Drug Administration (FDA) for the prevention of skeletal-related events in patients with bone metastases from solid tumors. Further clinical trials of denosumab for other bone remodeling related conditions are currently under way, i.e. for bone metastases from other forms of cancer (Lipton A et al. Randomized Active-Controlled Phase II Study of Denosumab Efficacy and Safety in Patients With Breast Cancer-Related Bone MetastasesJ Clin Oncol 25:4431-4437 (2007); Neville-Webbe H L, Coleman R E. Bisphosphonates and RANK ligand inhibitors for the treatment and prevention of metastatic bone disease. Eur J Cancer 2010; 46:1211-1222; Santini D, Galluzzo S, Zoccoli A, Pantano F, Fratto M E, et al. New molecular targets in bone metastases. Canc Treat Rev 2010; 36S3:S6-10).

It would therefore be desirable to have a therapeutic composition that is capable of binding to RANKL and is based on the naturally occurring OPG molecule, which, while having an acceptable pharmacological profile, has a broader therapeutic potential.

### SUMMARY OF THE INVENTION

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

In certain aspects, the present invention provides for pharmaceutical composition containing a polypeptide that binds human RANKL with a Kd value of no more than about 5×10⁻¹³M. The polypeptide comprises a first amino acid sequence comprising amino acids 1 through 215 of human osteoprotegerin (GenBank: U94332.1). The polypeptide further comprises a second amino acid sequence comprising amino acids 103 through 329 of human immunoglobulin gamma-1 Fc (GenBank: J00228.1). The polypeptide may comprise amino acid sequence of SEQ ID NO. 1.

In certain aspects, the present invention provides for a therapeutic composition. The composition comprises a polypeptide that binds to human RANKL. The polypeptide comprises a biologically active portion of human osteoprotegerin and a Fc portion of human immunoglobulin gamma-1. The polypeptide binds human RANKL with a Kd value of no more than about 5×10⁻¹³M.

The polypeptide may exhibit a half-life in systemic circulation in Cynomolgus monkey of at least 48 hours after a subcutaneous administration of the therapeutic composition at a dose of 3 mg/kg. The polypeptide may exhibit a half-life in systemic circulation in Cynomolgus monkey of at least 38 hours after a subcutaneous administration of the therapeutic composition at a dose of 10 mg/kg.
1. The therapeutic composition may also contain about 25 mM sodium phosphate, from about 50 mM to about 100 mM NaCl, from about 20 to about 25 mM L-Arginine hydrochloride, while having pH value from about 6.3 to about 6.8. The therapeutic composition may also contain about 10 mg/mL sucrose. The therapeutic composition may also contain from about 10 mg/mL to about 25 mg/mL mannitol.

In certain aspects, the present invention provides for a use of a substance for manufacture of a medicament for the treatment or prevention of a disease associated with bone resorption or remodeling. The substance comprises a polypeptide that binds to human RANKL. The polypeptide comprises a first amino acid sequence comprising amino acids 1 through 215 of human osteoprotegerin. The polypeptide further comprises a second amino acid sequence comprising amino acids 103 through 329 of human immunoglobulin gamma-1 Fc. The first amino acid sequence in the polypeptide may precede the second amino acid sequence. The polypeptide may comprise amino acid sequence of SEQ ID NO. 1. The disease associated with bone resorption or remodeling may be a carcinoma, a breast cancer, a prostate cancer, multiple myeloma, a bone sarcoma, bone metastases due to solid tumors, osteoporosis, rheumatoid arthritis, or psoriatic arthritis.

In certain aspects, the present invention provides for a method of treating or preventing a disease associated with bone resorption or remodeling. The method comprises administering to a patient in need for treating or preventing a disease associated with bone resorption or remodeling a therapeutically effective amount of a pharmaceutical composition comprising a polypeptide that binds to human RANKL. The polypeptide comprises a first amino acid sequence comprising amino acids 1 through 215 of human osteoprotegerin. The polypeptide further comprises a second amino acid sequence comprising amino acids 103 through 329 of human immunoglobulin gamma-1 Fc. The first amino acid sequence in the polypeptide may precede the second amino acid sequence. The polypeptide may comprise amino acid sequence of SEQ ID NO. 1. The disease associated with bone resorption or remodeling may be a carcinoma, a breast cancer, a prostate cancer, multiple myeloma, a bone sarcoma, bone metastases due to solid tumors, osteoporosis, rheumatoid arthritis, or psoriatic arthritis.

These and other aspects and advantages of the invention described herein will become apparent upon consideration of the Figures and detailed description below.

### BRIEF DESCRIPTION OF THE DRAWINGS

The following drawings and descriptions are provided to aid in the understanding of the invention:

FIG. 1 shows association and dissociation curves of Denosumab at various rshRANKL concentrations generated by BIAcore X100;

FIG. 2 shows association and dissociation curves of polypeptide of SEQ ID NO. 1 at different RANKL concentrations generated by BIAcore X100;

FIG. 3 shows representative size-exclusion (SEC) HPLC chromatograms of polypeptide of SEQ ID NO. 1 analyzed at 0 days time point (panel A), 67 days time point (panel B) and 176 days time point (panel C);

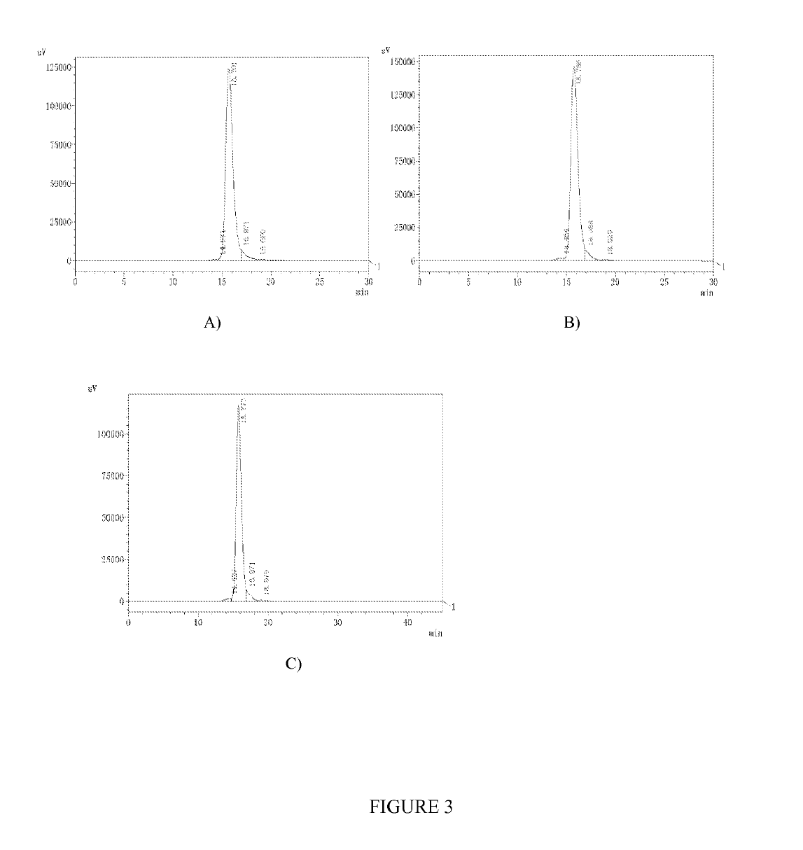

FIG. 4 shown primate single polypeptide of SEQ ID NO. 1 dose linearity for Cₘₐₓ and AUCₗₐₛₜ for administered doses of 0.3, 3, 10 and 30 mg/kg for the subcutaneous route of administration; and

FIG. 5 shown primate single polypeptide of SEQ ID NO. 1 dose study results corrected by the dose for Cₘₐₓ and AUCₗₐₛₜ for administered doses of 0.3, 3, 10 and 30 mg/kg for the subcutaneous route of administration.

### DETAILED DESCRIPTION OF THE INVENTION

The teachings disclosed herein are based, in part, upon engineering of a protein molecule comprising a biologically active N-terminal portion of OPG which is fused to the Fc portion of a human IgG. To enable recombinant production of such OPG-derived protein molecule, a DNA expression vector has been constructed for overproducing the protein molecule in a heterologous protein expression system, and mammalian cells have been prepared stably expressing the protein molecule to a high expression level. Design, preparation and preliminary characterization of composition of matter of the present teachings are disclosed, in part, in an International Patent Application Publication No. WO/2013/147899, published on Oct. 3, 2013, which is incorporated herein by reference in the entirety.

The protein molecule from the recombinant source formed homo-dimmers and homo-tetramers in solution. A protein purification procedure has been devised allowing obtaining a physiologically relevant substantially pure homo-dimeric preparation of the protein molecule. Unexpectedly, purified protein molecule demonstrates an exceptionally high degree of binding affinity for RANKL in an in vitro binding assay. Pharmaceutical formulations were devised allowing subcutaneous and intravenous administration of the protein molecule into primates. Thus formulated protein molecule exhibits an acceptable pharmacokinetics profile upon subcutaneous and intravenous administration into primates. Even further, thus formulated protein molecule exhibits substantial systemic exposure upon subcutaneous administration into humans.

The terms used in this specification generally have their ordinary meanings in the art, within the context of this invention and in the specific context where each term is used. Certain terms are discussed below or elsewhere in the specification, to provide additional guidance to the practitioner in describing the compositions and methods of the invention and how to make and use them. The scope or meaning of any use of a term will be apparent from the specific context in which the term is used. “About” and “approximately” shall generally mean an acceptable degree of error for the quantity measured given the nature or precision of the measurements. Typically, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The methods of the invention may include steps of comparing sequences to each other, including wild-type sequence to one or more mutants (sequence variants). Such comparisons typically comprise alignments of polymer sequences, e.g., using sequence alignment programs and/or algorithms that are well known in the art (for example, BLAST, FASTA and MEGALIGN, to name a few). The skilled artisan can readily appreciate that, in such alignments, where a mutation contains a residue insertion or deletion, the sequence alignment will introduce a “gap” (typically represented by a dash, or “A”) in the polymer sequence not containing the inserted or deleted residue.

The methods of the invention may include statistical calculations, e.g. determination of IC50 or EC50 values, etc. The skilled artisan can readily appreciate that such can be performed using a variety of commercially available software, e.g. PRISM (GraphPad Software Inc, La Jolla, Calif., USA) or similar.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions. However, in common usage and in the instant application, the term “homologous,” when modified with an adverb such as “highly,” may refer to sequence similarity and may or may not relate to a common evolutionary origin.

The term “sequence similarity,” in all its grammatical forms, refers to the degree of identity or correspondence between nucleic acid or amino acid sequences that may or may not share a common evolutionary origin.

The terms “protein” and “polypeptide” are used interchangeably. In general, OPG-derived proteins of the present teachings for use in mammals are expressed in mammalian cells that allow for proper post-translational modifications, such as CHO or HEK293 cell lines, although other mammalian expression cell lines are expected to be useful as well. It is therefore anticipated that the OPG-derived proteins may be post-translationally modified without substantially effecting their biological function.

In certain aspects, functional variants of OPG-derived protein molecules of the present teachings include fusion proteins having at least a biologically active portion of the human OPG and one or more fusion domains. Well known examples of such fusion domains include, but are not limited to, polyhistidine, Glu-Glu, glutathione S transferase (GST), thioredoxin, protein A, protein G, an immunoglobulin heavy chain constant region (e.g., an Fc), maltose binding protein (MBP), or human serum albumin. A fusion domain may be selected so as to confer a desired property. For example, the OPG polypeptide portion may be fused with a domain that stabilizes the OPG polypeptide in vivo (a “stabilizer” domain), optionally via a suitable peptide linker. The term “stabilizing” means anything that increases the half life of a polypeptide in systemic circulation, regardless of whether this is because of decreased destruction, decreased clearance, or other pharmacokinetic effect. Fusions with the Fc portion of an immunoglobulin are known to confer desirable pharmacokinetic properties on certain proteins. Likewise, fusions to human serum albumin can confer desirable properties. Other types of fusion domains that may be selected include multimerizing (e.g., dimerizing, tetramerizing) domains and functional domains that confer an additional biological function, e.g. promoting accumulation at the targeted site of action in vivo.

In certain aspects, the present invention provides for a polypeptide comprising the leading 215 amino acids of the human OPG (GenBank: U94332.1), followed by 227 amino acids of the Fc portion of the human Ig Gamma-1 (GenBank: J00228.1). In an example embodiment, the protein molecule of the present invention comprises amino acid sequence of SEQ ID NO. 1.

| hOPG-hIgG1-Fc polypeptide | |
|---|---|
| (SEQ ID NO. 1) | |
| MNKLLCCALV FLDISIKWTT QETFPPKYLH YDEETSHQLL CDKCPPGTYL KQHCTAKWKT | 60 |
| VCAPCPDHYY TDSWHTSDEC LYCSPVCKEL QYVKQECNRT HNRVCECKEG RYLEIEFCLK | 120 |
| HRSCPPGFGV VQAGTPERNT VCKRCPDGFF SNETSSKAPC RKHTNCSVFG LLLTQKGNAT | 180 |
| HDNICSGNSE STQKCGIDVT LCEEAFFRFA VPTKFDKTHT CPPCPAPELL GGPSVFLFPP | 240 |
| KPKDTLMISR TPEVTCVVVD VSHEDPEVKF NWYVDGVEVH NAKTKPREEQ YNSTYRVVSV | 300 |
| LTVLHQDWLN GKEYKCKVSN KALPAPIEKT ISKAKGQPRE PQVYTLPPSR DELTKNQVSL | 360 |
| TCLVKGFYPS DIAVEWESNG QPENNYKTTP PVLDSDGSFF LYSKLTVDKS RWQQGNVFSC | 420 |
| SVMHEALHNH YTQKSLSLSP GK | 442 |

In certain aspects, the present invention provides for a recombinant DNA molecule having an open reading frame coding for a polypeptide comprising the leading 215 amino acids of the human OPG followed by 227 amino acids of the Fc portion of the human Ig Gamma-1, optionally connected via a flexible linker. In an example embodiment, the recombinant DNA molecule of the present invention comprises nucleotide sequence of SEQ ID NO. 2.

| hOPG-hIgG1-Fc DNA | |
|---|---|
| (SEQ ID NO. 2) | |
| ATGAATAAGC TGCTGTGCTG TGCCCTCGTG TTTCTCGATA TAAGCATTAA GTGGACTACC | 60 |
| CAGGAGACAT TCCCTCCTAA GTATCTGCAC TATGACGAGG AGACAAGCCA TCAGCTGCTG | 120 |
| TGCGATAAGT GTCCTCCTGG GACCTATCTC AAACAACATT GTACAGCCAA ATGGAAGACA | 180 |
| GTCTGCGCTC CATGTCCTGA CCACTACTAC ACCGACTCTT GGCATACTAG CGACGAATGT | 240 |
| CTGTATTGTT CACCCGTGTG CAAGGAGCTG CAATACGTGA AACAGGAATG CAATAGGACA | 300 |
| CATAACCGCG TGTGTGAATG CAAAGAGGGC AGGTATCTGG AGATCGAATT TTGTCTGAAG | 360 |
| CACCGGAGCT GCCCACCCGG CTTTGGAGTG GTCCAGGCCG GGACTCCCGA GAGAAACACT | 420 |
| GTGTGCAAAA GATGCCCAGA CGGATTCTTT TCAAACGAGA CATCTTCTAA GGCACCATGT | 480 |
| CGGAAGCACA CTAACTGTTC CGTCTTTGGG CTGCTGCTCA CCCAGAAGGG CAATGCCACC | 540 |
| CACGATAATA TTTGCTCCGG AAACTCCGAA TCCACCCAAA AGTGCGGGAT AGATGTTACC | 600 |
| CTCTGCGAAG AGGCATTCTT CCGCTTCGCT GTTCCTACCA AGTTCGACAA AACTCACACA | 660 |
| TGCCCACCGT GCCCAGCTCC GGAACTCCTG GGCGGACCGT CAGTCTTCCT CTTCCCCCCA | 720 |
| AAACCCAAGG ACACCCTCAT GATCTCCCGG ACCCCTGAGG TCACATGCGT GGTGGTGGAC | 780 |
| GTGAGCCACG AAGACCCTGA GGTCAAGTTC AACTGGTACG TGGACGGCGT GGAGGTGCAT | 840 |
| AATGCCAAGA CAAAGCCGCG GGAGGAGCAG TACAACAGCA CGTACCGTGT GGTCAGCGTC | 900 |
| CTCACCGTCC TGCACCAGGA CTGGCTGAAT GGCAAGGAGT ACAAGTGCAA GGTCTCCAAC | 960 |
| AAAGCCCTCC CAGCCCCCAT CGAGAAAACC ATCTCCAAAG CCAAAGGGCA GCCCCGAGAA | 1020 |
| CCACAGGTGT ACACCCTGCC CCCATCCCGG GATGAGCTGA CCAAGAACCA GGTCAGCCTG | 1080 |
| ACCTGCCTGG TCAAAGGCTT CTATCCCAGC GACATCGCCG TGGAGTGGGA GAGCAATGGG | 1140 |
| CAGCCGGAGA ACAACTACAA GACCACGCCT CCCGTGTTGG ACTCCGACGG CTCCTTCTTC | 1200 |
| CTCTACAGCA AGCTCACCGT GGACAAGAGC AGGTGGCAGC AGGGGAACGT CTTCTCATGC | 1260 |
| TCCGTGATGC ATGAGGCTCT GCACAACCAC TACACGCAGA AGAGCCTCTC CCTGTCTCCG | 1320 |
| GGTAAA | 1326 |

In certain aspects, the present invention provides for a recombinant mammalian expression plasmid for high expression of a polypeptide comprising the leading 215 amino acids of the human OPG followed by 227 amino acids of the Fc portion of the human Ig Gamma-1, optionally connected via a flexible linker. This plasmid comprises the cytomegalovirus (CMV) promoter to drive transcription of the gene coding for said polypeptide, followed by the bGH polyadenylation and transcription termination sequence. The plasmid also contains a pUC origin of replication and β-lactamase gene, which confers ampicillin resistance, for supporting plasmid propagation and selection in bacteria. The plasmid further contains a gene for Glutamine synthetase, a selectable marker widely used for establishing stable CHOK1 and NSO cell lines.

In an example embodiment, the mammalian expression plasmid of the present invention comprises nucleotide sequence of SEQ ID NO. 3.

| hOPG-hIgG1-Fc expression plasmid | |
|---|---|
| (SEQ ID NO. 3) | |
| GAATTCATTG ATCATAATCA GCCATACCAC ATTTGTAGAG GTTTTACTTG CTTTAAAAAA | 60 |
| CCTCCCACAC CTCCCCCTGA ACCTGAAACA TAAAATGAAT GCAATTGTTG TTGTTAACTT | 120 |
| GTTTATTGCA GCTTATAATG GTTACAAATA AAGCAATAGC ATCACAAATT TCACAAATAA | 180 |
| AGCATTTTTT TCACTGCATT CTAGTTGTGG TTTGTCCAAA CTCATCAATG TATCTTATCA | 240 |
| TGTCTGGCGG CCGCGAGACG CCATCCACGC TGTTTTGACC TCCATAGAAG ACACCGGGAC | 300 |
| CGATCCAGCC TCCGCGGCCG GGAACGGTGC ATTGGAACGC GGATTCCCCG TGCCAAGAGT | 360 |
| GACGTAAGTA CCGCCTATAG AGTCTATAGG CCCACCCCCT TGGCTTCTTA TGCATGCTAT | 420 |
| ACTGTTTTTG GCTTGGGGTC TATACACCCC CGCTTCCTCA TGTTATAGGT GATGGTATAG | 480 |
| CTTAGCCTAT AGGTGTGGGT TATTGACCAT TATTGACCAC TCCCCTATTG GTGACGATAC | 540 |
| TTTCCATTAC TAATCCATAA CATGGCTCTT TGCCACAACT CTCTTTATTG GCTATATGCC | 600 |
| AATACACTGT CCTTCAGAGA CTGACACGGA CTCTGTATTT TTACAGGATG GGGTCTCATT | 660 |
| TATTATTTAC AAATTCACAT ATACAACACC ACCGTCCCCA GTGCCCGCAG TTTTTATTAA | 720 |
| ACATAACGTG CTCCACGCGA ATCTCGGGTA CGTGTTCCGG ACATGGGCTC TTCTCCGGTA | 780 |
| GCGGCGGAGC TTCTACATCC GAGCCCTGCT CCCATGCCTC CAGCGACTCA TGGTCGCTCG | 840 |
| GCAGCTAGTG GAGGCCAGAC TTAGGCACAG CACGATGCCC ACCACCACCA GTGTGCCGCA | 900 |
| CAAGGCCGTG GCGGTAGGGT ATGTGTCTGA AAATGAGCTC GGGGAGCGGG CTTGCACCAA | 960 |
| AAATTTTCGC GTCGACTATA CCGTCCACAT GTGAGCAAAA GGCCAGCAAA AGGCCAGGAA | 1020 |
| CCGTAAAAAG GCCGCGTTGC TGGCGTTTTT CCATAGGCTC CGCCCCCCTG ACGAGCATCA | 1080 |
| CAAAAATCGA CGCTCAAGTC AGAGGTGGCG AAACCCGACA GGACTATAAA GATACCAGGC | 1140 |
| GTTTCCCCCT GGAAGCTCCC TCGTGCGCTC TCCTGTTCCG ACCCTGCCGC TTACCGGATA | 1200 |
| CCTGTCCGCC TTTCTCCCTT CGGGAAGCGT GGCGCTTTCT CATAGCTCAC GCTGTAGGTA | 1260 |
| TCTCAGTTCG GTGTAGGTCG TTCGCTCCAA GCTGGGCTGT GTGCACGAAC CCCCCGTTCA | 1320 |
| GCCCGACCGC TGCGCCTTAT CCGGTAACTA TCGTCTTGAG TCCAACCCGG TAAGACACGA | 1380 |
| CTTATCGCCA CTGGCAGCAG CCACTGGTAA CAGGATTAGC AGAGCGAGGT ATGTAGGCGG | 1440 |
| TGCTACAGAG TTCTTGAAGT GGTGGCCTAA CTACGGCTAC ACTAGAAGAA CAGTATTTGG | 1500 |
| TATCTGCGCT CTGCTGAAGC CAGTTACCTT CGGAAAAAGA GTTGGTAGCT CTTGATCCGG | 1560 |
| CAAACAAACC ACCGCTGGTA GCGGTTTTTT TGTTTGCAAG CAGCAGATTA CGCGCAGAAA | 1620 |
| AAAAGGATCT CAAGAAGATC CTTTGATCTT TTCTACGGGG TCTGACGCTC AGTGGAACGA | 1680 |
| AAACTCACGT TAAGGGATTT TGGTCATGAG ATTATCAAAA AGGATCTTCA CCTAGATCCT | 1740 |
| TTTAAATTAA AAATGAAGTT TTAAATCAAT CTAAAGTATA TATGAGTAAA CTTGGTCTGA | 1800 |
| CAGTTACCAA TGCTTAATCA GTGAGGCACC TATCTCAGCG ATCTGTCTAT TTCGTTCATC | 1860 |
| CATAGTTGCC TGACTCCCCG TCGTGTAGAT AACTACGATA CGGGAGGGCT TACCATCTGG | 1920 |
| CCCCAGTGCT GCAATGATAC CGCGAGACCC ACGCTCACCG GCTCCAGATT TATCAGCAAT | 1980 |
| AAACCAGCCA GCCGGAAGGG CCGAGCGCAG AAGTGGTCCT GCAACTTTAT CCGCCTCCAT | 2040 |
| CCAGTCTATT AATTGTTGCC GGGAAGCTAG AGTAAGTAGT TCGCCAGTTA ATAGTTTGCG | 2100 |
| CAACGTTGTT GCCATTGCTA CAGGCATCGT GGTGTCACGC TCGTCGTTTG GTATGGCTTC | 2160 |
| ATTCAGCTCC GGTTCCCAAC GATCAAGGCG AGTTACATGA TCCCCCATGT TGTGCAAAAA | 2220 |
| AGCGGTTAGC TCCTTCGGTC CTCCGATCGT TGTCAGAAGT AAGTTGGCCG CAGTGTTATC | 2280 |
| ACTCATGGTT ATGGCAGCAC TGCATAATTC TCTTACTGTC ATGCCATCCG TAAGATGCTT | 2340 |
| TTCTGTGACT GGTGAGTACT CAACCAAGTC ATTCTGAGAA TAGTGTATGC GGCGACCGAG | 2400 |
| TTGCTCTTGC CCGGCGTCAA TACGGGATAA TACCGCGCCA CATAGCAGAA CTTTAAAAGT | 2460 |
| GCTCATCATT GGAAAACGTT CTTCGGGGCG AAAACTCTCA AGGATCTTAC CGCTGTTGAG | 2520 |
| ATCCAGTTCG ATGTAACCCA CTCGTGCACC CAACTGATCT TCAGCATCTT TTACTTTCAC | 2580 |
| CAGCGTTTCT GGGTGAGCAA AAACAGGAAG GCAAAATGCC GCAAAAAAGG GAATAAGGGC | 2640 |
| GACACGGAAA TGTTGAATAC TCATACTCTT CCTTTTTCAA TATTATTGAA GCATTTATCA | 2700 |
| GGGTTATTGT CTCATGAGCG GATACATATT TGAATGTATT TAGAAAAATA AACAAATAGG | 2760 |
| GGTTCCGCGC ACATTTCCCC GAAAAGTGCC ACCTGACCTC GACGGATCGG GAGATCTCCC | 2820 |
| GATCCCCTAT GGTGCACTCT CAGTACAATC TGCTCTGATG CCGCATAGTT AAGCCAGTAT | 2880 |
| CTGCTCCCTG CTTGTGTGTT GGAGGTCGCT GAGTAGTGCG CGAGCAAAAT TTCTGTGGAA | 2940 |
| TGTGTGTCAG TTAGGGTGTG GAAAGTCCCC AGGCTCCCCA GCAGGCAGAA GTATGCAAAG | 3000 |
| CATGCATCTC AATTAGTCAG CAACCAGGTG TGGAAAGTCC CCAGGCTCCC CAGCAGGCAG | 3060 |
| AAGTATGCAA AGCATGCATC TCAATTAGTC AGCAACCATA GTCCCGCCCC TAACTCCGCC | 3120 |
| CATCCCGCCC CTAACTCCGC CCAGTTCCGC CCATTCTCCG CCCCATGGCT GACTAATTTT | 3180 |
| TTTTATTTAT GCAGAGGCCG AGGCCGCCTC TGCCTCTGAG CTATTCCAGA AGTAGTGAGG | 3240 |
| AGGCTTTTTT GGAGGCCTAG GCTTTTGCAA AAAGCTAAGC TACAACAAGG CTCTGGCTAA | 3300 |
| CTAGAGAACC CACTGCTTAC TGGCTTATCG AAAGCTAGCT TAATACGACT CAATGAATCA | 3360 |
| GGGTGCAAAC AAGACGGTAT TAGACCGATA TTTACGGTTA GATATCCCGG ACCAGAAATG | 3420 |
| TCAAGCTATG TACATCTGGG TCGATGGAAC CGGCGAAAAC CTCCGCTCTA AGACCAGGAC | 3480 |
| ACTCAACTTT ACTCCTAAAT CTCCCAGTGA GCTGCCAATA TGGAATTTCG ATGGGTCATC | 3540 |
| AACGGGCCAG GCCGAACGGA GCAACAGTGA CGTGTACCTG TATCCAGTCG CTGTTTATCG | 3600 |
| AGATCCATTC AGGCTGGGTA ACAATAAGCT GGTCCTCTGT GAAACCTACA AATACAACAA | 3660 |
| GAAGCCTGCT GATACTAACC AGCGTTGGAA GTGTATGGAA GTAATGACAA GGGCAGCAGA | 3720 |
| CCAGCACCCA TGGTTCGGCA TGGAACAAGA ATATACTCTT TTGGACATTG ACAAACATCC | 3780 |
| CTTGGGTTGG CCCAAGAATG GCTATCCAGG CCCTCAGGGT CCCTATTACT GTGGTGTGGG | 3840 |
| TGCTAATAGG GTGTATGGGC GCGATGTGGT CGAGGCTCAC TACAGGGCGT GTCTTTGCGC | 3900 |
| TGGCATCAAC ATCTCTGGGG AGAACGCGAA AGTCATGCCG GCCCAATGGG AGTTCCAGGT | 3960 |
| TGGTCCGTGT GAAGGCATAA CCATGGGCGA CGACCTCTGG ATGGCTCGCT ACCTTCTTCA | 4020 |
| CAGGGTCGCT GAGGACTTTG ATGTTGTAGT AACACTCGAC CCCAAGCCAA TCCCTGGTGA | 4080 |
| CTGGAACGGC GCTGGAATGC ACACTAATTT CTCTACTGAA GCCATGCGTG GTCCCAATGG | 4140 |
| CATTCTGGAA ATTGAGAGTG CCATCGACAA ATTGTCGAAG GTTCATGAGA AACACATCAA | 4200 |
| GGCATACGAC CCACACGCAG GCAAGGATAA CGAAAGGCGC TTGACTGGTC ATTATGAAAC | 4260 |
| TTCCTCCATC CATGACTTTT CTGCAGGTGT GGCCAACCGT GGTGCCTCCA TCCGCATCCC | 4320 |
| CAGAGGAGTG GCTGAGGAGA AAACCGGCTA CCTGGAGGAC CGTCGCCCTT CCTCCAACGC | 4380 |
| TGACCCTTAT GTGGTGTCTG AGAGGCTTGT GCGTACCATC TGCCTGAACG AGCAGTGACT | 4440 |
| ATAGGGAGAC CCAAGCTGAC GCGCCCTGTA GCGGCGCATT AAGCGCGCCC GGGCTGGTGG | 4500 |
| TTACGCGCAG CGTGACCGCT ACACTTGCCA GCGCCCTAGC GCCCGCTCCT TTCGCTTTCT | 4560 |
| TCCCTTCCTT TCTCGCCACG TTCGCCGGCT TTCCCCGTCA AGCTCTAAAT CGGGGGCTCC | 4620 |
| CTTTAGGGTT CCGATTTAGT GCTTTACGGC ACCTCGACCC CAAAAAACTT GATTAGGGTG | 4680 |
| ATGGTTCACG TAGTGGGCCA TCGCCCTGAT AGACGGTTTT TCGCCCTTTG ACGTTGGAGT | 4740 |
| CCACGTTCTT TAATAGTGGA CTCTTGTTCC AAACTGGAAC AACACTCAAC CCTATCTCGG | 4800 |
| TCTATTCTTT TGATTTATAA GGGATTTTCT CTAGCTAGAG CTTGGCGTAA TCATGGTCAT | 4860 |
| AGCTGTTTCC TGTGTGAAAT TGTTATCCGC TCACAATTCC ACACAACATA CGAGCCGGAA | 4920 |
| GCATAAAGTG TAAAGCCTGG GGTGCCTAAT GAGTGAGCTA ACTCACATTA ATTGCGTTGC | 4980 |
| GCTCACTGCC CGCTTTCCAG TCGGGAAACC TGTCGTGCCA GCTGCATTAA TGAATCGGCC | 5040 |
| AACGCGCGGG GAGAGGCGGT TTGCGTATTG GGCGCTCTTC CGCTTCCTCG CTGTCCACCT | 5100 |
| CGCTGCGCTC GGTCGTTCGG CTGCGGCGAG CGGTATCAGC TCACTCAAAG GCGGTAATAC | 5160 |
| GGTTAGAGAT TTCGATTCCA CCGCCGCCTT CTATGAAAGG TTGGGCTTCG GAATCGTTTT | 5220 |
| CCGGGACGCC GGCTGGATGA TCCTCCAGCG CGGGGATCTC ATGCTGGAGT TCTTCGCCCA | 5280 |
| CCCCAACTTG TTTATTGCAG CTTATAATGG TTACAAATAA AGCAATAGCA TCACAAATTT | 5340 |
| CACAAATAAA GCATTTTTTT CACTGCATTC TAGTTGTGGT TTGTCCAAAC TCATCAATGT | 5400 |
| ATCTTATCAT GTCTGTATAC CGTCGACCTC AAGGCTTGAC CGACAATTGC ATGAAGACGC | 5460 |
| GTAATCTGCT TAGGGTTAGT TTTACAGGAT GGGGTCTCAT TTATTATTTA CAAATTCACA | 5520 |
| TATACAACAC CACCAGATCG CCTGGAGACG CCATCCACGC TGTTTTGACC TCCATAGAAG | 5580 |
| ACACCGGGAC CGATCCAGCC TCCGCGGCCG GGAACGGTGC ATTGGAACGC GGATTCCCCG | 5640 |
| TGCCAAGAGT GACGTAAGTA CCGCCTATAG AGTCTATAGG CCCACCCCCT TGGCTTCTTA | 5700 |
| TGCATGCTAT ACTGTTTTTG GCTTGGGGTC TATACACCCC CGCTTCCTCA TGTTATAGGT | 5760 |
| GATGGTATAG CTTAGCCTAT AGGTGTGGGT TATTGACCAT TATTGACCAC TCCCCTATTG | 5820 |
| GTGACGATAC TTTCCATTAC TAATCCATAA CATGGCTCTT TGCCACAACT CTCTTTATTG | 5880 |
| GCTATATGCC AATACACTGT CCTTCAGAGA CTGACACGGA CGCGTTTTGC GCTGCTTCGC | 5940 |
| GATGTACGGG CCAGATATAC GCGTTGACAT TGATTATTGA CTAGTTATTA ATAGTAATCA | 6000 |
| ATTACGGGGT CATTAGTTCA TAGCCCATAT ATGGAGTTCC GCGTTACATA ACTTACGGTA | 6060 |
| AATGGCCCGC CTGGCTGACC GCCCAACGAC CCCCGCCCAT TGACGTCAAT AATGACGTAT | 6120 |
| GTTCCCATAG TAACGCCAAT AGGGACTTTC CATTGACGTC AATGGGTGGA GTATTTACGG | 6180 |
| TAAACTGCCC ACTTGGCAGT ACATCAAGTG TATCATATGC CAAGTACGCC CCCTATTGAC | 6240 |
| GTCAATGACG GTAAATGGCC CGCCTGGCAT TATGCCCAGT ACATGACCTT ATGGGACTTT | 6300 |
| CCTACTTGGC AGTACATCTA CGTATTAGTC ATCGCTATTA CCATGGTGAT GCGGTTTTGG | 6360 |
| CAGTACATCA ATGGGCGTGG ATAGCGGTTT GACTCACGGG GATTTCCAAG TCTCCACCCC | 6420 |
| ATTGACGTCA ATGGGAGTTT GTTTTGGCAC CAAAATCAAC GGGACTTTCC AAAATGTCGT | 6480 |
| AACAACTCCG CCCCATTGAC GCAAATGGGC GGTAGGCGTG TACGGTGGGA GGTCTATATA | 6540 |
| AGCAGAGCTC GTTTAGTGAA CCGTCAGATC GCCTGGAGAC GCCATCCACG CTGTTTTGAC | 6600 |
| CTCCATAGAA GACACCGGGA CCGATCCAGC CTCCGCGGCC GGGAACGGTG CATTGGAACG | 6660 |
| CGGATTCCCC GTGCCAAGAG TGACGTAAGT ACCGCCTATA GAGTCTATAG GCCCACCCCC | 6720 |
| TTGGCTTCTT ATGCATGCTA TACTGTTTTT GGCTTGGGGT CTATACACCC CCGCTTCCTC | 6780 |
| ATGTTATAGG TGATGGTATA GCTTAGCCTA TAGGTGTGGG TTATTGACCA TTATTGACCA | 6840 |
| CTCCCCTATT GGTGACGATA CTTTCCATTA CTAATCCATA ACATGGCTCT TTGCCACAAC | 6900 |
| TCTCTTTATT GGCTATATGC CAATACACTG TCCTTCAGAG ACTGACACGG ACTCTGTATT | 6960 |
| TTTACAGGAT GGGGTCTCAT TTATTATTTA CAAATTCACA TATACAACAC CACCGTCCCC | 7020 |
| AGTGCCCGCA GTTTTTATTA AACATAACGT GGGATCTCCA CGCGAATCTC GGGTACGTGT | 7080 |
| TCCGGACATG GGCTCTTCTC CGGTAGCGGC GGAGCTTCTA CATCCGAGCC CTGCTCCCAT | 7140 |
| GCCTCCAGCG ACTCATGGTC GCTCGGCAGC TCCTTGCTCC TAACAGTGGA GGCCAGACTT | 7200 |
| AGGCACAGCA CGATGCCCAC CACCACCAGT GTGCCGCACA AGGCCGTGGC GGTAGGGTAT | 7260 |
| GTGTCTGAAA ATGAGCTCGG GGAGCGGGCT TGCACCGCTG ACGCATTTGG AAGACTTAAG | 7320 |
| GCAGCGGCAG AAGAAGATGC AGGCAGCTGA GTTGTTGTGT TCTGATAAGA GTCAGAGGTA | 7380 |
| ACTCCCGTTG CGGTGCTGTT AACGGTGGAG GGCAGTGTAG TCTGAGCAGT ACTCGTTGCT | 7440 |
| GCCGCGCGCG CCACCAGACA TAATAGCTGA CAGACTAACA GACTGTTCCT TTCCATGGGT | 7500 |
| CTTTTCTGCA GTCACCGTCC TTGACACGAA GCTTGCCACC ATGAATAAGC TGCTGTGCTG | 7560 |
| TGCCCTCGTG TTTCTCGATA TAAGCATTAA GTGGACTACC CAGGAGACAT TCCCTCCTAA | 7620 |
| GTATCTGCAC TATGACGAGG AGACAAGCCA TCAGCTGCTG TGCGATAAGT GTCCTCCTGG | 7680 |
| GACCTATCTC AAACAACATT GTACAGCCAA ATGGAAGACA GTCTGCGCTC CATGTCCTGA | 7740 |
| CCACTACTAC ACCGACTCTT GGCATACTAG CGACGAATGT CTGTATTGTT CACCCGTGTG | 7800 |
| CAAGGAGCTG CAATACGTGA AACAGGAATG CAATAGGACA CATAACCGCG TGTGTGAATG | 7860 |
| CAAAGAGGGC AGGTATCTGG AGATCGAATT TTGTCTGAAG CACCGGAGCT GCCCACCCGG | 7920 |
| CTTTGGAGTG GTCCAGGCCG GGACTCCCGA GAGAAACACT GTGTGCAAAA GATGCCCAGA | 7980 |
| CGGATTCTTT TCAAACGAGA CATCTTCTAA GGCACCATGT CGGAAGCACA CTAACTGTTC | 8040 |
| CGTCTTTGGG CTGCTGCTCA CCCAGAAGGG CAATGCCACC CACGATAATA TTTGCTCCGG | 8100 |
| AAACTCCGAA TCCACCCAAA AGTGCGGGAT AGATGTTACC CTCTGCGAAG AGGCATTCTT | 8160 |
| CCGCTTCGCT GTTCCTACCA AGTTCGACAA AACTCACACA TGCCCACCGT GCCCAGCTCC | 8220 |
| GGAACTCCTG GGCGGACCGT CAGTCTTCCT CTTCCCCCCA AAACCCAAGG ACACCCTCAT | 8280 |
| GATCTCCCGG ACCCCTGAGG TCACATGCGT GGTGGTGGAC GTGAGCCACG AAGACCCTGA | 8340 |
| GGTCAAGTTC AACTGGTACG TGGACGGCGT GGAGGTGCAT AATGCCAAGA CAAAGCCGCG | 8400 |
| GGAGGAGCAG TACAACAGCA CGTACCGTGT GGTCAGCGTC CTCACCGTCC TGCACCAGGA | 8460 |
| CTGGCTGAAT GGCAAGGAGT ACAAGTGCAA GGTCTCCAAC AAAGCCCTCC CAGCCCCCAT | 8520 |
| CGAGAAAACC ATCTCCAAAG CCAAAGGGCA GCCCCGAGAA CCACAGGTGT ACACCCTGCC | 8580 |
| CCCATCCCGG GATGAGCTGA CCAAGAACCA GGTCAGCCTG ACCTGCCTGG TCAAAGGCTT | 8640 |
| CTATCCCAGC GACATCGCCG TGGAGTGGGA GAGCAATGGG CAGCCGGAGA ACAACTACAA | 8700 |
| GACCACGCCT CCCGTGTTGG ACTCCGACGG CTCCTTCTTC CTCTACAGCA AGCTCACCGT | 8760 |
| GGACAAGAGC AGGTGGCAGC AGGGGAACGT CTTCTCATGC TCCGTGATGC ATGAGGCTCT | 8820 |
| GCACAACCAC TACACGCAGA AGAGCCTCTC CCTGTCTCCG GGTAAATAAT A | 8871 |

In certain aspects, the present invention provides for a mammalian expression system for production of a polypeptide comprising the leading 215 amino acids of the human OPG followed by 227 amino acids of the Fc portion of the human Ig Gamma-1, optionally connected via a flexible linker The expression system of the present invention comprises a mammalian cell harboring a recombinant mamalian expression plasmid for high expression of a polypeptide comprising the leading 215 amino acids of the human OPG followed by 227 amino acids of the Fc portion of the human Ig Gamma-1, optionally connected via a flexible linker

In an example embodiment, the mammalian expression system of the present invention comprises Chinese hamster ovary cells (CHO-K1) harboring a plasmid comprising nucleotide sequence of SEQ ID NO. 3.

In certain aspects, the present invention provides for a method of treatment of a mammal effected by a disorder associated with bone resorption or remodeling.

### EXAMPLES

The following Examples illustrate the forgoing aspects and other aspects of the present invention. These non-limiting Examples are put forth so as to provide those of ordinary skill in the art with illustrative embodiments as to how the compounds, compositions, articles, devices, and/or methods claimed herein are made and evaluated. The Examples are intended to be purely exemplary of the invention and are not intended to limit the scope of what the inventor regard as his invention. Efforts have been made to ensure accuracy with respect to numbers (e.g., amounts, temperature, etc.) but some errors and deviations should be accounted for.

### Example 1

### Preparation of Polypeptides of the Present Invention

hOPG-hIgG1-Fc polypeptide of SEQ ID. 1 was expressed in CHO-K1 using molecular biology, cell culture and protein biochemistry techniques known in the art and described PCT Publication WO/2013/147899. Essentially, CHO-K1 cells expressing the polypeptide were harvested and lysed utilizing well established protocols. After cell lysate clarification, the supernatant containing expressed hOPG-hIgG1-Fc polypeptide was first applied to a Protein A affinity column. The pH adjusted Protein A column eluate was further purified by anion-exchange chromatography (AIEX) utilizing Q Sepharose resin. The AIEX flowthrough was analyzed by size-exclusion HPLC (SEC-HPLC), SDS-PAGE and other analytical techniques, as appropriate.

For subsequent studies, a therapeutic composition comprising hOPG-hIgG1-Fc polypeptide was formulated to contain 40 mg hOPG-hIgG1-Fc polypeptide in 4 mL also containing 1% sucrose, 100 mM sodium chloride, 20 mM L-arginine hydrochloride and 25 mM sodium phosphate pH 6.3. A single vial contains about 40 mg hIgG1-Fc polypeptide in a volume of 4 mL. Thus the protein concentration in a vial is 10±1 mg/mL.

### Example 2

### Evaluation of Polypeptides of the Present Teachings Affinity Binding to RANKL Using Surface Plasmon Resonance (SPR) Assay

The binding affinity of prepared hOPG-hIgG1-Fc polypeptide of SEQ ID NO. 1 to the recombinant soluble human Receptor Activator of Nuclear Factor Kappa-B Ligand (rshRANKL) was measured using a specially designed Surface Plasmon Resonance (SPR) assay. In the assay, hOPG-hIgG1-Fc polypeptide was captured on the SPR sensor surface by recombinant Protein A solutions of rshRANKL at different concentrations were dispensed over the sensor surface and kinetics of association and dissociation were monitored. Affinity is calculated by fitting a 1:1 Langmuir binding model to the data.

Materials, Reagents and Equipment:
Biacore CM5 Sensor Chip (GE Healthcare); Amine Coupling Kit (GE Healthcare); 50 mM sodium hydroxide (GE Healthcare); 10mM NaAcetate PH 4.5 (GE Healthcare); Surfactance P20 (GE Healthcare); Glycine (Sigma-Aldrich); 10×PBS Buffer (GE Healthcare)
Buffers:
Working Buffer: PBS+0.05% Surfactance P20 (pH7.26)
Regeneration Buffer: 5.4 ml of 10 mM Glycine buffer+4.6 ml of 10 mM Glycine buffer (pH 1.7)
Materials:
Denosumab (Prolia, commercial product, Amgen) concentration 60 mg/mL
hOPG-hIgG1-Fc polypeptide preparation per foregoing Example 1
rshRANKL concentration 0.76 mg/mL (13 μM)
Equipment:
Biacore X100 Instrument (GE Healthcare)
Biacore X100 Evaluation Software V2.0.1 (GE Healthcare)

Procedures:

Recombinant Protein A was diluted using pH4.5 10 mM NaAcetate buffer to a final concentration of 0.025 mg/ml. Protein A was immobilized on Flow Cells 1-2 using coupling kit and following parameters: a) 7 minute injection of 1:1 EDC: NHS; b) 5 minute injection of diluted Protein A in 10 mM NaAcetate pH4.5 at 10μL/min; c) 7 minute injection of 1M ethanolamine pH8.5. rshRANKL (MW 57.9 kDa) was dilute to desired concentrations with BIAcore working buffer. Five different rshRANKL dilutions were used for affinity measurements: 1.0415 nM, 2.083 nM, 4.166 nM, 8.333 nM, 16.666 nM. Six different rshRANKL dilutions were used for Denosumab affinity measurements: 0.7359 nM, 1.4719 nM, 2.94375 nM, 5.8875 nM, 11.775 nM, 23.55 nM. Denosumab was diluted using BIAcore working buffer to a final concentration of 0.8 μM. hOPG-hIgG1-Fc polypeptide sample was diluted using BIAcore working buffer to a final concentration of 37 nM

Assay Protocol:

The assay was performed according to the manufacturer's protocol Sample compartment temperature was 25° C.; data collection rate—1 Hz; flow rate—30μL/min; five different concentrations of rshRANKL were used for hOPG-hIgG1-Fc polypeptide evaluation (4.166 nM dilution was measured twice); six different concentrations of rshRANKL ligand were used for Denosumab evaluation. All measurements were performed as following: Denosumab/ hOPG-hIgG1-Fc capture with contact time of 180s; rshRANKL capture with contact time of 180s; dissociation using working buffer for 3600s; regeneration using regeneration buffer for 70s.

The Biacore X100 Evaluation software is used to estimate the kinetic association (kₐ) and dissociation (k_{d}) constants, the equilibrium dissociation constant (K_{D}) and the maximum RANKL binding level (Rₘₐₓ) for each sample. The model parameters are estimated for each sample individually by fitting a 1:1 Langmuir binding model to the data.

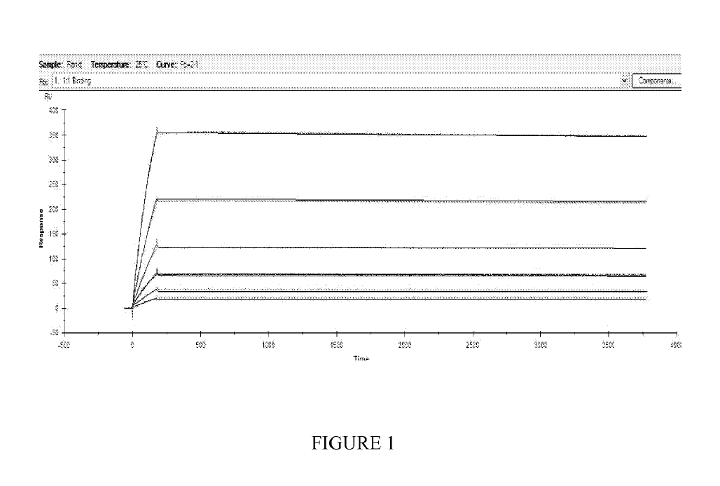

Association and dissociation curves of Denosumab at different RANKL concentrations are showed in FIG. 1. Correlated data for each curve is listed in Table 1. The model parameters of Denosumab-RANKL binding which were estimated via Biacore X100 Evaluation software are listed in Table 2.

The affinity (K_{D}) of anti-RANKL antibody Denosumab binding to RANKL is 2.6×10⁻¹¹M, which is consistent with reported data from the manufacturer.

TABLE 1
Correlated data for Denosumab-rshRANKL binding.

| Curve | | | Conc (M) | Flow (ul/min) | kt (RU/Ms) | RI (RU) |
|---|---|---|---|---|---|---|
| Cycle: 5 | 0.7359 | nM | 7.359E−10 | 30 | 1.07346E+18 | 2.729222012 |
| Cycle: 6 | 1.4719 | nM | 1.4719E−09 | 30 | 1.07346E+18 | 4.866918331 |
| Cycle: 7 | 2.94375 | nM | 2.94375E−09 | 30 | 1.07346E+18 | 6.830413213 |
| Cycle: 8 | 5.8875 | nM | 5.8875E−09 | 30 | 1.07346E+18 | 5.581231192 |
| Cycle: 9 | 11.775 | nM | 1.1775E−08 | 30 | 1.07346E+18 | 2.692233789 |
| Cycle: 10 | 23.55 | nM | 2.355E−08 | 30 | 1.07346E+18 | −1.728466535 |

TABLE 2
Model parameters used for estimating Denosumab-RANKL binding.

| kₐ | k_{d} | K_{D} | Rₘₐₓ | | Chi² | |
|---|---|---|---|---|---|---|
| (1/Ms) | (1/s) | (M) | (RU) | t_{c} | (RU²) | U-value |
| 235360.1515 | 6.21706E−06 | 2.64151E−11 | 562.403892 | 3.45471E+17 | 8.62705 | 11.64904 |

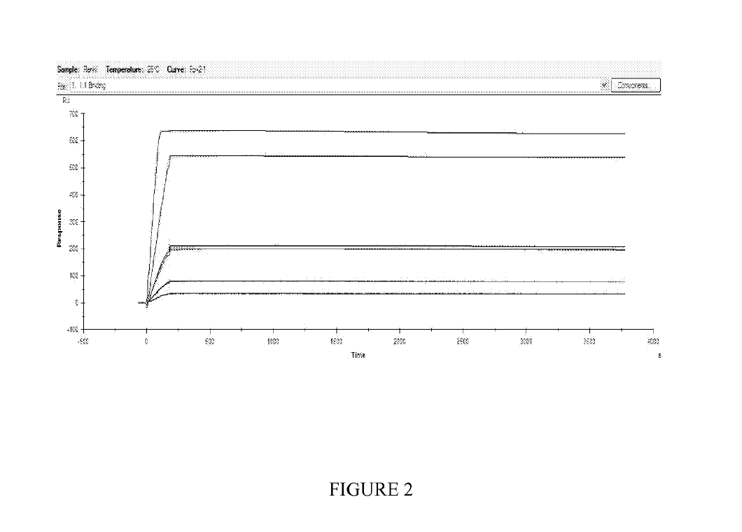

Association and dissociation curves of hOPG-hIgG1-Fc polypeptide at different rshRANKL concentrations are showed in FIG. 2. Correlated data for each curve is listed in Table 3. The model parameters of hOPG-hIgG1-Fc-RANKL binding estimated by Biacore X100 Evaluation software are listed in the Table 4.

The affinity (K_{D}) of hOPG-hIgG1-Fc binding to RANKL is 4.85×10⁻¹³M.

TABLE 3
Correlated data for each curve of hOPG-hIgG1-Fc-RANKL binding.

| Curve | | | Rmax (RU) | Conc (M) | Flow (ul/min) | RI (RU) | kt (RU/Ms) |
|---|---|---|---|---|---|---|---|
| Cycle: 5 | 1.0415 | nM | 44.96516 | 1.04E−09 | 30 | −1.0584 | 4.33E+08 |
| Cycle: 6 | 2.083 | nM | 87.28024 | 2.08E−09 | 30 | −6.8384 | 4.33E+08 |
| Cycle: 7 | 4.166 | nM | 205.2188 | 4.17E−09 | 30 | −23.1182 | 4.33E+08 |
| Cycle: 8 | 8.333 | nM | 567.9522 | 8.33E−09 | 30 | −29.2618 | 4.33E+08 |
| Cycle: 9 | 16.666 | nM | 639.3375 | 1.67E−08 | 30 | −4.20587 | 4.33E+08 |
| Cycle: 11 | 4.166 | nM | 218.8427 | 4.17E−09 | 30 | −20.7052 | 4.33E+08 |

TABLE 4
Model parameters of hOPG-hIgG1-Fc-RANKL binding estimated via Biacore X100.

| kₐ (1/Ms) | k_{d} (Vs) | K_{D} (M) | t_{c} | Chi² (RU²) | U-value |
|---|---|---|---|---|---|
| 13476795.16 | 6.54E−06 | 4.85E−13 | 1.39E+08 | 5.541974 | 5.30225 |

Thus, the affinity (K_{D}) of hOPG-hIgG1-Fc polypeptide of SEQ ID NO. 1 binding to rhsRANKL was estimated to be about 4.9×10⁻¹³ M, which is approximately 50 times higher compared to that of commercially available Denosumab, which under substantially similar experimental conditions was estimated to be about 2.6×10⁻¹¹M.

### Example 3

### Formulation Stability Study of hOPG-hIgG1-Fc Polypeptide

hOPG-hIgG1-Fc polypeptide of SEQ ID NO. 1 was expressed and purified essentially as described in the forgoing. Long-term stability study of the hOPG-hIgG1-Fc polypeptide performed to estimate product stability at 2-8° C. hOPG-hIgG1-Fc polypeptide accelerated stability study at 40° C. was performed to evaluate product stability in formulation buffers of the different compositions. The polypeptide stability was analyzed by SEC HPLC. Integration of the SEC HPLC chromatograms was performed to evaluate hOPG-hIgG1-Fc polypeptide monomers, aggregates and degradation products and to monitor the changes in the protein composition. hOPG-hIgG1-Fc polypeptide was aliquoted into screw capped vials. The aliquots were stored in the dark at designated temperatures during required periods of time.

Materials and Equipment:

All reagents used were at least HPLC grade: Milli-Q Water (or equivalent); Sodium Chloride (J T Baker); Sodium Phosphate Dibasic, Heptahydrate (Na₂HPO₄.7H₂O, J T Baker) or Sodium Phosphate Dibasic Anhydrous (Na₂HPO₄, J T Baker); 6 N Hydrochloric Acid (J T Baker); Sodium Hydroxide 6N NaOH (BDH); Sodium Azide (Sigma Aldrich); Methanol (J T Baker); rhsRANKL (Alphamab, Inc.); goat anti-human IgG:HRP conjugate, (Perkin-Elmer).

pH Meter (Corning Pinnacle 542); Analytical Balance (Mettler Toledo XS603S); Waters HPLC System with PDA and Empower Software; YMC-Pack Diol 300, 6.0 mm ID×30 cm, (YMC Catalog Number DL06S053006WT); G2000 SW×1, 7.5 mm×300 mm (TOSOH Bioscience); TSK Guard SW, 7.5 mm×75 mm (TOSOH Bioscience); Inline Filter with 2 μm frit (VWR Catalog Number 21511-442); Replacement 2 μm Frit (VWR Catalog Number 21511-423); Filter, PES, 1000 mL (Nalgene, Catalog Number 567-0020); Total Recovery Vial, screw top 12×32 mm cap with PTFE/Silicone septa (Waters); Cap/Septa 12×32 screw neck with bonded pre-slit PTFE/Silicone septa (Waters).

Buffers:

Mobile Phase buffers:
100 mM NaPhosphate, 200 mM NaCl pH 7.0 (PBS) for YMC-Pack Diol 300 column
20 mM NaPhosphate, 300 mM NaCl, pH 7.4 buffer. Filtered and degassed for G2000SW×1 column

Following drug formulation buffers were prepared:
1. 25 mM NaPhosphate, 100 mM NaCl, 25 mM L-Arginine HCl, 10 mg/mL Sucrose, pH 6.3
2. 25 mM NaPhosphate, 100 mM NaCl, 25 mM L-Arginine HCl, 10 mg/mL Sucrose, pH 6.8
3. 20 mM Histidine, 100 mM NaCl, 25 mM L-Arginine HCl, 10 mg/mL Mannitol, pH 6.8
4. 20 mM Histidine, 50 mM NaCl, 25 mM L-Arginine HCl, 25 mg/mL Mannitol, pH 6.8
5. 20 mM Histidine, 50 mM NaCl, 25 mM L-Arginine HCl, 25 mg/mL Mannitol, 0.1 mg/mL Methionine, pH 6.8
6. 20 mM Histidine, 100 mM NaCl, 25 mM L-Arginine HCl, 10 mg/mL Mannitol, pH 6.3
7. 20 mM Histidine, 50 mM NaCl, 25 mM L-Arginine HCl, 25 mg/mL Mannitol, pH 6.3
8. 20 mM Histidine, 50 mM NaCl, 25 mM L-Arginine HCl, 25 mg/mL Mannitol, 0.1 mg/mL Methionine, pH 6.3

Procedures:

Samples were diluted with mobile phase to reach protein concentration of 1 mg/ml or 2 mg/mL.

Chromatography parameters:

Flow Rate: 0.5 ml/min

Column Temperature: 25±3° C.

Autosampler Temperature: 5±3° C.

Injection Volume: 15 μl for samples with 2 mg/mL polypeptide concentration
25 μl for samples with 1 mg/mL polypeptide concentration

Detector Wavelength: 280 nm for samples with 2 mg/mL polypeptide concentration
214 nm for samples with 1 mg/mL polypeptide concentration

Run Time: 35 min

Two lots of hOPG-hIgG1-Fc polypeptide individual preparations were tested, both at about 10 mg/ml total protein concentration formulated in 25 mM NaPhosphate, 100 mM NaCl, 25 mM L-Arginine HCl, 10 mg/mL Sucrose, at pH 6.3.

Results of the 2-8° C. stability studies for two different preparations lots of hOPG-hIgG1-Fc polypeptide are summarized in Table 5 and Table 6 below.

TABLE 5
hOPG-hIgG1-Fc polypeptide composition
at the stability study time points

| | | Time points (days) | | |
|---|---|---|---|---|
| Sample | Composition | 0 | 67 | 176 |
| Lot No 1 | Monomer (%) | 94.3 | 94.4 | 93.9 |
| | Aggregates (%) | 0.6 | 1.3 | 1.3 |
| | Degradation products (%) | 5.1 | 4.3 | 4.8 |

TABLE 6
hOPG-hIgG1-Fc polypeptide composition
at the stability study time points

| | | Time points | | | |
|---|---|---|---|---|---|
| Sample | Composition | 1 day | 4 weeks | 9 weeks | 12 weeks |
| Lot No 2 | Monomer (%) | 98.1 | 97.2 | 97.2 | 97.1 |
| | Aggregates (%) | 0.38 | 0.43 | 0.2 | 0.56 |
| | Degradation | 1.5 | 2.4 | 2.6 | 2.4 |
| | products (%) | | | | |

Preparation Lot No 2 was subjected accelerated stability tests at 40° C. in formulation buffers of various compositions. Formulation buffer of Lot No 2 preparation (25 mM NaPhosphate, 100 mM NaCl, 25 mM L-Arginine HCl, 10 mg/mL Sucrose, pH 6.3) was exchanged with the drug formulation buffers listed in the forgoing disclosure. Results of the accelerated stability study are summarized in Table 7 below.

TABLE 7
hOPG-hIgG1-Fc polypeptide composition at the accelerated
stability study time points (formulation buffers F1-F8)

| | | Time points (days) | | | | |
|---|---|---|---|---|---|---|
| Sample | Composition | 0 | 7 | 14 | 21 | 28 |

F1-25 mM NaPhosphate, 100 mM NaCl, 25 mM L-Arginine HCl, 10 mg/mL Sucrose, pH 6.3

| | | | | | | |
|---|---|---|---|---|---|---|
| Lot No 2 | Monomer (%) | 96.6 | 94.2 | 91.4 | 87.3 | 83.7 |
| | Aggregates (%) | 0.8 | 1.6 | 3.2 | 4.9 | 6.3 |
| | Degradation products (%) | 2.6 | 4.2 | 5.4 | 7.8 | 10.0 |

F2-25 mM NaPhosphate, 100 mM NaCl, 25 mM L-Arginine HCl, 10 mg/mL Sucrose, pH 6.8

| | | | | | | |
|---|---|---|---|---|---|---|
| Lot No 2 | Monomer (%) | 96.8 | 92.6 | 89.1 | 83.4 | 83.7 |
| | Aggregates (%) | 0.6 | 2.1 | 4.1 | 6.3 | 6.3 |
| | Degradation products (%) | 2.6 | 5.3 | 6.8 | 10.3 | 10.0 |

F3-20 mM Histidine, 100 mM NaCl, 25 mM L-Arginine HCl, 10 mg/mL Mannitol, pH 6.8

| | | | | | | |
|---|---|---|---|---|---|---|
| Lot No 2 | Monomer (%) | 96.6 | 94.2 | 91.3 | 88.3 | 84.8 |
| | Aggregates (%) | 0.6 | 1.5 | 2.4 | 3.2 | 3.7 |
| | Degradation products (%) | 2.8 | 4.3 | 6.3 | 8.5 | 11.5 |

F4-20 mM Histidine, 50 mM NaCl, 25 mM L-Arginine HCl, 10 mg/mL Mannitol, pH 6.8

| | | | | | | |
|---|---|---|---|---|---|---|
| Lot No 2 | Monomer (%) | 96.6 | 94.3 | 91.2 | 88.2 | 86.4 |
| | Aggregates (%) | 0.6 | 1.6 | 2.4 | 3.8 | 3.8 |
| | Degradation products (%) | 2.8 | 4.1 | 6.4 | 8.0 | 9.8 |

F5-20 mM Histidine, 50 mM NaCl, 25 mM L-Arginine HCl,
25 mg/mL Mannitol, 0.1 mg/mL Methionine, pH 6.8

| | | | | | | |
|---|---|---|---|---|---|---|
| Lot No 2 | Monomer (%) | 96.8 | 94.3 | 85.8 | 87.7 | 86.7 |
| | Aggregates (%) | 0.6 | 1.5 | 2.1 | 2.3 | 3.1 |
| | Degradation products (%) | 2.6 | 4.2 | 12.1 | 10.0 | 10.2 |

F6-20 mM Histidine, 50 mM NaCl, 25 mM L-Arginine HCl, 10 mg/mLMannitol, pH 6.3

| | | | | | | |
|---|---|---|---|---|---|---|
| Lot No 2 | Monomer (%) | 96.8 | 94.4 | 93.4 | 88.7 | 88.1 |
| | Aggregates (%) | 0.6 | 1.4 | 1.9 | 2.3 | 2.2 |
| | Degradation products (%) | 2.6 | 4.2 | 4.7 | 9.0 | 9.7 |

F7-20 mM Histidine, 100 mM NaCl, 25 mM L-Arginine HCl, 25 mg/mL Mannitol, pH 6.3

| | | | | | | |
|---|---|---|---|---|---|---|
| Lot No 2 | Monomer (%) | 96.8 | 94.4 | 93.0 | 91.2 | 87.9 |
| | Aggregates(%) | 0.6 | 1.4 | 1.8 | 3.0 | 2.9 |
| | Degradation products (%) | 2.6 | 4.2 | 5.2 | 5.8 | 7.2 |

F8-20 mM Histidine, 50 mM NaCl, 25 mM L-Arginine HCl,
25 mg/mL Mannitol, 0.1 mg/mL Methionine, pH 6.3

| | | | | | | |
|---|---|---|---|---|---|---|
| Lot No 2 | Monomer (%) | 96.4 | 95.0 | 92.8 | 91.0 | 88.6 |
| | Aggregates (%) | 0.6 | 1.3 | 1.8 | 2.1 | 2.0 |
| | Degradation products (%) | 3.0 | 3.7 | 5.4 | 6.9 | 9.4 |

As is apparent from the results summarized in Table 7, addition of mannitol into formulation buffer up to 10 mg/mL improved hOPG-hIgG1-Fc polypeptide composition stability, however pH increase of the formulation buffer from 6.3 to 6.8 did not affect product stability, neither did changes in NaCl concentration.

Stability of Lot No 2 preparation was also tested at room temperature (RT). The preparation was diluted with 0.9% NaCl into three samples having total protein concentrations of 0.6, 1.2, 1.8 mg/mL, respectively. The samples were stored at room temperature for 24 h, and analyzed at 0 hr time point and 24 hr time point. hOPG-hIgG1-Fc polypeptide composition stability was analyzed with SEC HPLC, which monitors integrity of protein composition and ELISA, to assess the binding of hOPG-hIgG1-Fc to RANKL with acceptance criteria of 70-130% of the reference standard binding to RANKL. The results of the study are summarized in Table 8.

TABLE 8
hOPG-hIgG1-Fc polypeptide composition at the RT stability study time points

| | Control 10 mg/mL | | 0.6 mg/mL | | 1.2 mg/mL | | 1.8 mg/mL | |
|---|---|---|---|---|---|---|---|---|
| | 0 hr | 24 hr | 0 hr | 24 hr | 0 hr | 24 hr | 0 hr | 24 hr |
| Monomer (SEC-HPLC) | 95.12% | 94.63% | 94.19% | 93.56% | 95.08% | 94.29% | 95.04% | 94.57% |
| Binding Activity | 94.9% | — | 88.8% | 94.10% | 89.3% | 79.40% | 88.6% | 115.60% |
| (ELISA) | | | | | | | | |

As is apparent from the results summarized in Table 8, hOPG-hIgG1-Fc polypeptide 10 mg/ml stock diluted to 0.6 mg/mL, 1.2 mg/mL and 1.8 mg/mL and stored at room temperature for 24 hours demonstrated integrity of composition and binding activity similar to the reference standard. Therefore, the data confirmed stability of the post-reconstituted hOPG-hIgG1-Fc polypeptide solutions during time sufficient for the drug preparation and intravenous administration.

Long-term stability of Lot No 2 preparation was tested at 2-8° C. The results of the study are summarized in Table 9.

TABLE 9
hOPG-hIgG1-Fc polypeptide 2-8° C. long term stability study results

| Assays | Test Method | Specification | T = 0 | T = 3 months | T = 6 months |
|---|---|---|---|---|---|
| Appearance | 999-GMP-064 | Clear, colorless, free | Clear, colorless, free | Clear, colorless, free | Clear, colorless, free of |
| | | of visible particles | visible particles | of visible particles | of visible particles |
| pH | 999-GMP-017 | 6.1-6.5 | 6.4 | 6.4 | 6.4 |
| A280 | 774-01-001 | 9.0-11.0 mg/mL | 10.1 mg/mL | 10.8 mg/mL | 10.0 mg/mL |
| Coomassie | 774-01-004 | Conforms to | Conforms to reference | Conforms to reference | Conforms to |
| Reduced | | reference standard | standard | standard | reference standard |
| Coomassie | 774-01-004 | Conforms to | Conforms to reference | Conforms to reference | Conforms to |
| Non-Reduced | | reference standard | standard | standard | reference standard |
| RankL ELISA | 774-01-009 | 70-130% | 100% | 89% | 81% |
| IEF | 774-01-007 | FIO, report pl range | 16 bands; pl range 6.0- | 11 bands; pl range 6.0- | 11 bands; pl range |
| | | and number of bands | 8.0 | 7.8 | 6.0-7.8 |
| SEC-HPLC | 774-01-002 | ≧92% monomer | 96% monomer | 97% monomer | 97% monomer |
| Osmolality | 999-GMP-024 | 280-350 mOsm/kg | 298 mOsm/kg | 301 mOsm/kg | 300 mOsm/kg |
| Endotoxin | 774-01-028 | ≦0.25 EU/mg | <0.04 EU/mg | N/A | N/A |
| Bioburden | 999-GMP-484 | ≦3 CFU/10 mL with | <1 CFU/10 mL with no | N/A | N/A |
| | | no objectionable | objectionable | | |
| | | organisms | organisms | | |

As is apparent from the results summarized in Table 8, hOPG-hIgG1-Fc polypeptide of SEQ ID NO. 1 at 10 mg/ml, formulated in 25 mM NaPhosphate, 100 mM NaCl, 25 mM L-Arginine HCl, 10 mg/mL Sucrose, at pH 6.3, maintains structural stability and specific activity at least for 6 months.

### Example 4

### Single Dose Pharmacokinetics Study of hOPG-hIgG1-Fc Polypeptide in Primates

Pharmacokinetic profile and the maximum tolerated dose of hOPG-hIgG1-Fc polypeptide following subcutaneous and intravenous (bolus) administration were studied in the 12 Cynomolgus monkeys (6 males and 6 females) that received single dose of hOPG-hIgG1-Fc polypeptide via subcutaneous administration at dose levels of 0.3, 3, 10, 30 and 100 mg/kg or via intravenous administration at dose levels of 0.3, 3 and 10 mg/kg. Subcutaneous dosing was done at a dose volume of 1 mL/kg, whilst intravenous dosing was done at a dose volume of 2 mL/kg. The test substance concentration was 9.73 mg/mL, for higher dose levels the dose volume were adjusted (based on animals weight) to achieve the target dose level/s.

The following vehicle was used for the preparation of the test substance to the required dose concentrations; formulation buffer (1% w/v Sucrose, 100 mM Sodium Chloride, 20 mM L-Arginine Hydrochloride, 25 mM Sodium Bicarbonate, final adjusted pH of 6.3). The vehicle was stored refrigerated and used within one week after preparation.

The animals were approximately 2-4 years and weighed 2-4 kg at the time of study commencement. The animals were observed twice daily for clinical signs. Body weight was recorded prior to each dose escalation. Food consumption was visually assessed daily during the study. Blood samples for clinical pathology investigations of haematology and clinical chemistry were collected once pre-trial and 24 h after each dose level.

The animals were initially allocated to 2 dose groups and treated as follows:

| | Dose | Dose | Dose | | |
|---|---|---|---|---|---|
| Route of | Level | Concentration | Volume | No. of Animals | |
| Administration | (mg/kg) | (mg/mL) | (mL/kg) | Male | Female |
| Subcutaneous | 0.3 | 0.3 | 1 | 1 | 1 |
| injection | 3 | 3 | 1 | 1 | 1 |
| | 10 | 9.73 | 1 | 1 | 1 |
| Intravenous | 0.3 | 0.15 | 2 | 1 | 1 |
| (bolus) | | | | | |
| injection | | | | | |

The animals were subsequently dosed as follows:

| | Dose | Dose | Dose | | |
|---|---|---|---|---|---|
| Route of | Level | Concentration | Volume | No. of Animals | |
| Administration | (mg/kg) | (mg/mL) | (mL/kg) | Male | Female |
| Subcutaneous | 10 | 4.85 | 2.06 | 1* | 1* |
| injection | 30 | 9.73 | 3.08 | 1** | 1** |
| Intravenous | 3 | 1.5 | 2 | 1* | 1* |
| (bolus) injection | 10 | 5 | 2 | 1** | 1** |

*Animals previously dosed with hOPG-hIgG1-Fc polypeptide at 0.3 mg/kg
**Naïve animals

Additional subcutaneous dosing for 2 animals was done to evaluate the liver enzyme activity (i.e. Aspartate Aminotransferase, Alanine Aminotransferase and Lactate Dehydrogenase), dosing was as follows:

| | Dose | Dose | Dose | | |
|---|---|---|---|---|---|
| Route of | Level | Concentration | Volume | No. of Animals | |
| Administration | (mg/kg) | (mg/mL) | (mL/kg) | Male | Female |
| Subcutaneous | 10 | 4.85 | 2.06 | 1*** | 1*** |
| injection | | | | | |

***Animals previously dosed with hOPG-hIgG1-Fc polypeptide at 3.0 mg/kg via subcutaneous route

Two animals received a single subcutaneous injection of 100 mg/kg followed by a 2 week post dose observation period. Blood samples were collected for liver enzyme activity.

| | Dose | Dose | Dose | | |
|---|---|---|---|---|---|
| Route of | Level | Concentration | Volume | No. of Animals | |
| Administration | (mg/kg) | (mg/mL) | (mL/kg) | Male | Female |
| Subcutaneous | 100 | 9.73 | 10.3 | 1 | 1 |
| injection | | | | | |

Both animals were previously dosed with hOPG-hIgG1-Fc polypeptide at 0.3 and 10 mg/kg via subcutaneous route and at 0.3 and 3 mg/kg via intravenous route, respectively.

Blood samples for pharmacokinetic investigations were obtained at designated time points at each dose escalation.

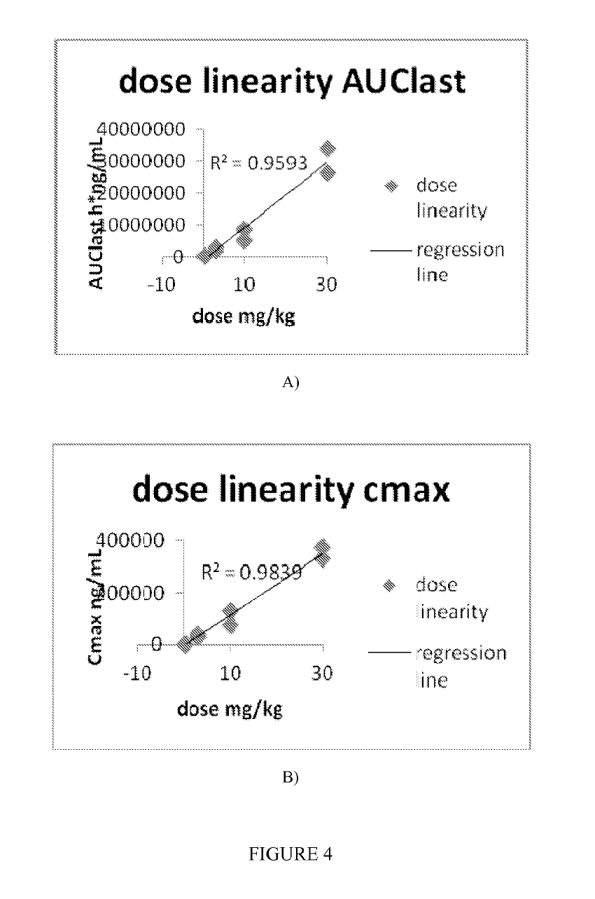

A pharmacokinetic analysis on hOPG-hIgG1-Fc polypeptide plasma levels was undertaken using a non-compartmental method using Phoenix™ for WinNonlin® (version 6.1, from Pharsight Corporation). C₀ (extrapolated concentration at T=0 For IV administration), Cmax (maximal concentration for SC administration) were obtained from the observed individuals values. AUCₗₐₛₜ (Area Under the Curve to the last data point) were determined by mixed logarithmic-linear regression. Results from the PK data showed the Cₘₐₓ of hOPG-hIgG1-Fc polypeptide to be approximately 6-8 h following subcutaneous administration at dose levels of 0.3, 3, 10 and 30 mg/kg, and approximately 1 h following intravenous injection at dose levels of 0.3 and 3 and 10 mg/kg. Clearance of hOPG-hIgG1-Fc polypeptide was approximately 168 h post dose for both intravenous and subcutaneous routes of administrations. For the SC route, hOPG-hIgG1-Fc polypeptide was not always immediately absorbed as showed by a T_{lag} of 1 h and 2 h observed at the lowest dose (0.3 mg/kg) and for animal 0590 (3 mg/kg), respectively. The T_{half} values indicated that the elimination of hOPG-hIgG1-Fc polypeptide was relatively slow with a T_{half} of about 55 h and 45 h for the IV and SC route, respectively, based on the 3 and 10 mg/kg doses values. For the IV route the T_{half} ranged from 27.5 h to 57.8 h and ranged from 35.5 h to 53.3 h for the SC route. The elimination rate showed no concomitant increase with dose rate. The elimination was similar for the both routes. For the IV and SC routes, the Cl and V_{d} were similar irrespective of the administered dose. For the IV route, the Cl and V_{d} ranged between 0.75 and 1.22 mL/h/kg and between 48.3 and 76.6 mL/kg, respectively. The corresponding ranges, for the SC route, were between 0.97 to 1.69 mL/h/kg and between 65.3 and 94.6 mL/kg. Cl and V_{d} did not increase with the increasing dose. These results showed that the T_{half}, Cl and V_{d} were independent of the administered dose. The Linearity of exposure for hOPG-hIgG1-Fc was only determined graphically for the SC route, from Cmax and AUClast by linear regression and these are presented in FIG. 4. Exposure increased linearly, R²=0.96 and 0.98 for AUCₗₐₛₜ and Cₘₐₓ, respectively.

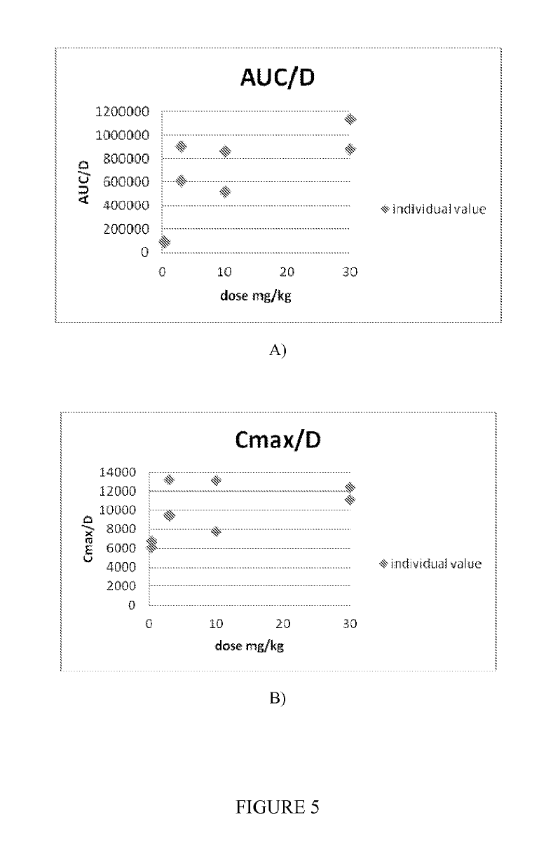

For the SC route, based the Cₘₐₓ values, it appeared that the dose proportionality can be showed (FIG. 5). The mean Cmax corrected by the dose were 6410, 11309, 10409 and 11739 (ng/mL)/(mg/kg) for 0.3, 3, 10 and 30 mg/kg doses, respectively. The dose proportionality was clearly demonstrated from 3 to 0.3 mg/kg doses. The exposure increased more than proportionality between 0.3 mg/kg dose in comparison to the 3 others. Actually, for a 10 fold increase in dose between 0.3 and 3 mg/kg, for a 33.3 fold increase between 0.3 and 10 mg/kg and for a100 fold increase in dose between 0.3 and 30 mg/kg, there were 17.6, 54.1 and 183.1 fold increase in Cₘₐₓ, respectively. The corresponding values for AUCₗₐₛₜ were 82.3, 249.5 and 1095.1, respectively which showed that the exposure increased for about 10 fold more than the dose ratios.

Between 3 and 10 mg/kg, relative dose proportionality was showed based on the Cₘₐₓ/D and AUCₗₐₛₜ /D and was confirmed by the dose ratio calculation which was closed to the theoretical dose ratios.

For the IV route, the exposure increased slightly more than the dose proportionality, for a 3.3 fold increase in dose there was a 4.3 and 4.1 increased in AUCₗₐₛₜ and Cₘₐₓ, respectively.

Thus, AUCₗₐₛₜ and Cₘₐₓ increased linearly (R²≈1.0) with a relatively dose-proportional exposure only from 3 to 30 mg/kg. These results showed that the systemic exposure increased after SC administration almost proportionally with the increasing dose from 3 to 30 mg/kg.

hOPG-hIgG1-Fc polypeptide bioavailability was estimated using the mean values obtained at 3 and 10 mg/kg and represented 88.6% and 59.2% at 3 and 10 mg/kg, respectively. It seemed that hOPG-hIgG1-Fc polypeptide had a better bioavialability at 3 mg/kg (88.6%) in comparison to 10 mg/kg (59.2%).

Thus, as is apparent from the results of the study, all animals were exposed to hOPG-hIgG1-Fc polypeptide at both administration routes evaluated and at all administered doses. After SC administration, systemic exposure to hOPG-hIgG1-Fc polypeptide (AUCₗₐₛₜ and Cₘₐₓ) increased linearly with the increasing dose with relatively dose-proportional exposure only from 3 to 30 mg/kg. The elimination rate was not affected by the increasing dose and was similar for the both routes. The Cl and V_{d} were similar irrespective of the administered dose whatever the administration route. The results showed that the T_{half}, Cl and Vd were independent of the administered dose. The results suggest that hOPG-hIgG1-Fc polypeptide had a better bioavailability at 3 mg/kg (88.6%) in comparison to 10 mg/kg (59.2%).

Mean plasma pharmacokinetic parameters for hOPG-hIgG1-Fc polypeptide after single subcutaneous and intravenous doses in primates are summarized in Table 9 below.

TABLE 9
Summary of mean plasma pharmacokinetic parameters for hOPG-hIgG1-Fc
polypeptide after single subcutaneous and intravenous doses in primates

| | | | | AUC | Cmax | T_{1/2} | CL | V |
|---|---|---|---|---|---|---|---|---|
| Species | Route | Dose | | (ng.h/mL) | (ng/mL) | (h) | (mL/h/kg) | (mL/kg) |
| Cynomolgus | SC | 0.3 | mg/kg | 27651 | 1923 | NC* | NC | NC |
| monkeys | | 3 | mg/kg | 2276384 | 33928 | 48.2 | 1.25 | 84.5 |
| | | 10 | mg/kg | 6899231 | 104091 | 38.9 | 1.38 | 76.0 |
| | | 30 | mg/kg | 30278949 | 352175 | NC | NC | NC |
| Cynomolgus | IV | 3 | mg/kg | 2647530 | 112132 | 42.6 | 1.07 | 62.4 |
| monkeys | | 10 | mg/kg | 11256849 | 460319 | 54.9 | 0.78 | 61.5 |

*NC—Not calculated

### Example 5

### Repeat-Dose Pharmacokinetics Study of hOPG-hIgG1-Fc Polypeptide in Primates

A repeat-dose pharmacokinetic analysis was completed during the toxicokinetic study in Cynomolgous monkeys, where four groups of 3 males and 3 females each from the main and the recovery group were treated twice weekly for 2 successive weeks by subcutaneous injection at concentrations of 0, 0.3, 3 and 10 mg/kg (n=3 animals per sex per group). Toxicokinetic analysis was undertaken only on animals administered the hOPG-hIgG1-Fc polypeptide.

A toxicokinetic analysis on plasma levels was undertaken using a non-compartmental method (for each day of kinetics, on Day 1 and on Day 13) using Phoenix™ for WinNonlin® (version 6.1, from Pharsight Corporation). Thus, the pharmacokinetic parameters were compared after administrations done on Days 1 and 13, respectively.

The study demonstrated the following. No quantifiable concentrations were detected in the control group (Group 1) even when hOPG-hIgG1-Fc polypeptide were measured in one male and one female of this group. All animals were exposed to hOPG-hIgG1-Fc polypeptide twice weekly for 2 weeks at the doses of 0.3, 3 and 10 mg/kg. Systemic exposure to hOPG-hIgG1-Fc polypeptide (mean AUC₇₂ and mean Cₘₐₓ) increased with the increasing dose in both males and females. Moderate accumulation of hOPG-hIgG1-Fc polypeptide was observed after a 2-week administration. The administration period did not depend on the administered dose levels. The Cₘₐₓ and AUC₇₂ increased following repeat administration at all dose levels in both males and females, as indicated by the ratio of AUC₇₂ between Day 13 and Day 1. This ratio ranged from 0.28 to 2.50 confirming a modest plasma accumulation of hOPG-hIgG1-Fc polypeptide. The accumulation increased with the administered dose level. No gender effect was showed higher than the intra-individual variability. No clear gender difference can be concluded and it can be considered that there was no difference between males and females. With respect to gender differences, the exposures were similar in both males and females across the different doses from 0.3 to 10 mg/kg. On day 1 and day 13, the systemic plasma exposure of hOPG-hIgG1-Fc polypeptide increased more than dose-proportionally between 0.3 and 10 mg/kg in both genders. No dose proportionality was observed, except on day 1, for females between the intermediate and the low doses in regards of the AUC₇₂ and for males between the high and low dose based on the Cₘₐₓ. On day 13, the increase of plasma exposure was at least 5 fold higher than the targeted dose ratio at the high level.

Mean plasma pharmacokinetic parameters for hOPG-hIgG1-Fc polypeptide after repeat-dose subcutaneous administration in primates are summarized in Table 10 below.

TABLE 10
Summary of mean plasma pharmacokinetic parameters in male
and female Cynomolgous monkeys (N = 3 per sex per dose
group) for hOPG-hIgG1-Fc polypeptide after twice weekly
subcutaneous dosing for two weeks

| | Dose (mg/kg) | | | | | |
|---|---|---|---|---|---|---|
| | 0.3 | | 3 | | 10 | |
| | Occasion | | | | | |
| | Day 1 | | | | | |
| | Sex | | | | | |
| | Male | Female | Male | Female | Male | Female |
| AUC₇₂ | 452067 | 235964 | 2673970 | 3203140 | 14821856 | 15224149 |
| (ng · | | | | | | |
| h/mL) | | | | | | |
| Cₘₐₓ | 14197 | 5365 | 45623 | 52601 | 233410 | 250589 |
| (ng/mL) | | | | | | |
| Tₘₐₓ (h) | 38.00 | 8.00 | 24.00 | 24.00 | 72.00 | 24.00 |
| AUC₇₂ | 1.92 | | 0.83 | | 0 97 | |
| (M/F) | | | | | | |
| Cₘₐₓ | 2.65 | | 0.87 | | 0.93 | |
| (M/F) | | | | | | |
| Dose | 10 (I/L) | | 3.33 (H/I) | | 33.3 (H/L) | |
| Ratio | | | | | | |
| AUC₇₂ | 5.91 | 13.57 | 5.54 | 4.75 | 32.8 | 64.5 |
| Ratio | | | | | | |
| Cₘₐₓ | 3.21 | 9.80 | 5.12 | 4.76 | 16.44 | 46.71 |
| Ratio | | | | | | |
| | Occasion | | | | | |
| | Week 2 (Day 13) | | | | | |
| AUC₇₂ | 189783 | 210980 | 4638633 | 6785492 | 33486023 | 35935204 |
| (ng · | | | | | | |
| h/mL) | | | | | | |
| Cₘₐₓ | 4262 | 3965 | 74853 | 110957 | 547783 | 606545 |
| (ng/mL) | | | | | | |
| Tₘₐₓ (h) | 8.00 | 8.00 | 24.00 | 8.00 | 8.00 | 24.00 |
| Dose | 10 (I/L) | | 3.33 (H/I) | | 33.3 (H/L) | |
| Ratio | | | | | | |
| AUC₇₂ | 24.44 | 32.16 | 7.22 | 5.30 | 176.4 | 170.3 |
| Ratio | | | | | | |
| Cₘₐₓ | 17.56 | 27.98 | 7.32 | 5.47 | 128.51 | 152.96 |
| Ratio | | | | | | |
| R_{ac} (day | 0.28 | 1.07 | 1.76 | 2.11 | 2.50 | 2.39 |
| 13/day 1) | | | | | | |

### Example 6

### Single Dose Pharmacokinetics Study of hOPG-hIgG1-Fc Polypeptide in Humans

Pharmacokinetic profile of hOPG-hIgG1-Fc polypeptide of SEQ ID NO. 1 following subcutaneous administration were studied in healthy male volunteers (ages 19-39) who received single dose of hOPG-hIgG1-Fc polypeptide via subcutaneous administration at dose levels of 10 mg, 30 mg and 60 mg. The drug formulation of hOPG-hIgG1-Fc polypeptide used was at about 10mg/ml total protein concentration formulated in 25 mM NaPhosphate, 100 mM NaCl, 25 mM L-Arginine HCl, 10 mg/mL Sucrose, at pH 6.3. The blood levels hOPG-hIgG1-Fc polypeptide at various time points post administration for the three doses tested are summarized in Tables 11-13. The results indicate a substantial post-subcutaneous administration bioavailability of the drug substance under study in systemic circulation in human subjects.

TABLE 11
Blood levels of hOPG-hIgG1-Fc polypeptide in healthy volunteers
after SC administration of a single 10 mg dose

| Time Point | hOPG-hIgG1-Fc polypeptide (ng/mL) per subject ID | | | | | | | |
|---|---|---|---|---|---|---|---|---|
| (Hours) | Ctrl 1 | Ctrl 2 | A** | B | C | D | E | F |
| 0 | 0 | 0 | 0 | 0 | 0 | 0 | 0 | 0 |
| 0.5 | 0 | 0 | 0 | 0 | 0 | 0 | 0 | 0 |
| 1 | 0 | 0 | 0 | 0 | 0 | 0 | 0 | 0 |
| 2 | 0 | 0 | 0 | 0 | 0 | 0 | 0 | 52.6 |
| 4 | 0 | 0 | 0 | 18.1 | 24.7 | 0 | 32.1 | 98.2 |
| 8 | 0 | 0 | 0 | 37.1 | 69.3 | 17.5 | 196.4 | 120.5 |
| 9 | 0 | 0 | 0 | 32.5 | 72.3 | 17.8 | 198.3 | 116.6 |
| 10 | 0 | 0 | 0 | 31.6 | 86.3 | 22.9 | 215.2 | 121.7 |
| 11 | 0 | 0 | 0 | 28.7 | 76.2 | 18.4 | 221.9 | 113.7 |
| 12 | 0 | 0 | 0 | 35.7 | 78.4 | 23.1 | 234.4 | 97.0 |
| 16 | 0 | 0 | 0 | 29.5 | 0 | 64.9 | 220.5 | 91.6 |
| 24 | 0 | 0 | 0 | 24.5 | 105.5 | 35.5 | 215.9 | 95.3 |
| 48 | 0 | 0 | 0 | 52.5 | 97.1 | 48.2 | 167.4 | 123.2 |
| 168 | 0 | 0 | 0 | 36.1 | 53.4 | 49.9 | 67.0 | 84.4 |
| 288 | 0 | 0 | 0 | 26.6 | 26.0 | 28.6 | 35.8 | 49.1 |
| 480 | 0 | 0 | 0 | 15.2 | 0 | 0 | 0 | 45.5 |
| 648 | 0 | 0 | 0 | 0 | 0 | 0 | 0 | 40.0 |

* 0.0 denotes that value obtained is below Lowest Limit of Quantification (1.683 ng/mL)
**Values were obtained for all time points, however were all below Lowest Limit of Quantification (1.683 ng/mL), thus cannot be reliably quantitated.

TABLE 12
Blood levels of hOPG-hIgG1-Fc polypeptide in healthy
volunteers after SC administration of a single 30 mg dose

| Time Point | hOPG-hIgG1-Fc polypeptide (ng/mL) per subject ID | | | | | | | |
|---|---|---|---|---|---|---|---|---|
| (Hours) | Ctrl 1 | Ctrl 2 | A | B | C | D | E | F |
| 0 | 0 | 0 | 0 | 0 | 0 | 0 | 0 | 0 |
| 0.5 | 0 | 0 | 0 | 0 | 0 | 0 | 0 | 0 |
| 1 | 0 | 0 | 0 | 24.8 | 17.7 | 34.1 | 0 | 0 |
| 2 | 0 | 0 | 0 | 67.2 | 340.4 | 37.7 | 17.6 | 0 |
| 4 | 0 | 0 | 44.6 | 160.2 | 1044.2 | 116.7 | 51.5 | 74.2 |
| 8 | 0 | 0 | 123.2 | 225.4 | 1940.2 | 288.4 | 92.2 | 187.0 |
| 9 | 0 | 0 | 143.3 | 198.9 | 2057.3 | 279.2 | 76.9 | 175.1 |
| 10 | 0 | 0 | 163.1 | 214.2 | 2085.1 | 352.7 | 93.2 | 221.6 |
| 11 | 0 | 0 | 167.6 | 194.4 | 2052.0 | 303.8 | 84.1 | 205.2 |
| 12 | 0 | 0 | 173.1 | 178.9 | 2331.4 | 353.6 | 77.8 | 205.7 |
| 16 | 0 | 0 | 196.6 | 156.9 | 2466.4 | 298.3 | 81.1 | 181.4 |
| 24 | 0 | 0 | 103.9 | 165.4 | 2321.4 | 262.6 | 109.4 | 171.3 |
| 48 | 0 | 0 | 168.1 | 164.3 | 1883.5 | 224.1 | 176.6 | 164.1 |
| 168 | 0 | 0 | 69.9 | 61.9 | 264.9 | 36.6 | 100.5 | 116.0 |
| 288 | 0 | 0 | 40.2 | 0 | 54.6 | 30.3 | 44.0 | 59.1 |
| 480 | 0 | 0 | 0 | 0 | 24.5 | 0 | 22.6 | 33.1 |
| 648 | 0 | 0 | 0 | 0 | 17.0 | 45.5 | 0 | 19.1 |

* 0.0 denotes that value obtained is below Lowest Limit of Quantification (1.683 ng/mL)

TABLE 13
Blood levels of hOPG-hIgG1-Fc polypeptide in healthy volunteers after SC administrsingle 60 mg dose

| Time Point | hOPG-hIgG1-Fc polypeptide (ng/mL) per subject ID | | | | | | | |
|---|---|---|---|---|---|---|---|---|
| (Hours) | Ctrl 1 | Ctrl 2 | A | B | C | D | E | F |
| 0 | 0 | 0 | 0 | 0 | 0 | 0 | 0 | 0 |
| 0.5 | 0 | 0 | 0 | 0 | 0 | 0 | 0 | 0 |
| 1 | 0 | 0 | 0 | 83.6 | 0 | 0 | 0 | 0 |
| 2 | 0 | 0 | 191.5 | 836.0 | 56.5 | 98.7 | 0 | 66.7 |
| 4 | 0 | 0 | 879.5 | 1818.6 | 222.9 | 370.8 | 276.3 | 275.7 |
| 8 | 0 | 0 | 1505.1 | 2963.0 | 376.7 | 567.1 | 579.3 | 686.7 |
| 9 | 0 | 0 | 1392.3 | 2744.2 | 365.4 | 632.7 | 719.7 | 739.3 |
| 10 | 0 | 0 | 1605.2 | 3054.6 | 450.6 | 720.5 | 732.5 | 1048.0 |
| 11 | 0 | 0 | 1672.7 | 3231.3 | 453.9 | 683.7 | 717.3 | 1028.3 |
| 12 | 0 | 0 | 1987.3 | 3293.0 | 434.6 | 708.4 | 700.6 | 1026.4 |
| 16 | 0 | 0 | 1860.1 | 3226.6 | 368.8 | 903.4 | 749.4 | 1180.0 |
| 24 | 0 | 0 | 1749.6 | 3252.6 | 547.9 | 834.8 | 718.1 | 1508.0 |
| 48 | 0 | 0 | 1849.4 | 3041.6 | 971.8 | 1119.4 | 1026.1 | 1766.0 |
| 168 | 0 | 0 | 523.7 | 1015.2 | 409.6 | 753.5 | 444.6 | 1804.0 |
| 288 | 0 | 0 | 251.1 | 339.6 | 113.0 | 174.7 | 182.3 | 575.5 |
| 480 | 0 | 0 | 81.5 | 0 | 41.7 | 62.7 | 84.1 | 321.5 |
| 648 | 0 | 0 | 43.5 | 0 | 37.6 | 0 | 71.1 | 180.6 |

*0.0 denotes that value obtained is below Lowest Limit of Quantification (1.683 ng/mL)

### Example 7

### Human Tissue Binding Characteristics Study of hOPG-hIgG1-Fc Polypeptide

The purpose of this immunohistochemistry (IHC) study is to determine tissue binding characteristics for hOPG-hIgG1-Fc polypeptide of SEQ ID NO. 1, and to compare the binding pattern to a commercially available therapeutic (Prolia). A commercially available TNF-alpha binding therapeutic etanercept (Enbrel) was used as an isotype control.

Methods

Titration experiments were conducted with 3 protein therapeutics, hOPG-hIgG1-Fc-FITC, PROLIA, and ENBREL (FITC (fluorescein isothiocyanate) labeling performed by Covance) to establish concentrations that would result in minimal background and maximal detection of signal.

Serial dilutions were performed at 20 μg/ml, 10 μg/ml, 5 μg/ml, and 2.5 μg/ml on fresh frozen human tissues supplied by LifeSpan. In addition ENBREL was further titered at 1.25 μg/ml, 0.6 μg/ml, and 0.3 μg/ml. hOPG-hIgG1-Fc-FITC, PROLIA-FITC, and the isotype control therapeutic ENBREL-FITC were used as the primary binding reagents, and the principal detection system consisted of an anti-FITC mouse secondary antibody (Sigma-Aldrich, catalog# F5636), followed by an anti-mouse secondary antibody (Vector, BA-2000), and a ABC-AP kit (AP=alkaline phosphatase secondary, Vector, AK-5000) with a Red substrate kit (Vector, SK-5100), which was used to produce a fuchsia-colored deposit. Tissues were also stained with positive control antibodies (CD31 and vimentin) to ensure that the tissue antigens were preserved and accessible for immunohistochemical analysis. Only tissues that were positive for CD31 and vimentin staining were selected for the remainder of the study. The negative controls consisted of performing the entire immunohistochemistry procedure on adjacent sections in the absence of primary reagents, or in the absence of both the primary reagent and the anti-FITC secondary antibody (using the anti-mouse tertiary and all other downstream reagents in all cases). The slides were interpreted by a pathologist and each reagent was evaluated for the presence of specific signal, level of background, and concordance with expression results reported in the literature. Staining intensity was recorded on a 0-4 scale (0=negative, 1=blush, 2=faint, 3=moderate, 4=strong). Slides were imaged with a DVC 1310C digital camera coupled to a Nikon microscope. Experimental results are summarized in Table 14 below.

TABLE 14
Results of the IHC study in human tissue

| | | | | | | CD31/ | | hOPG-hIgG1 | PROLIA | ENBREL | No Primary |
|---|---|---|---|---|---|---|---|---|---|---|---|
| Sam- | | | | Path- | | Vimentin | | 5 ug/ml | 2.5 ug/ml | 1.25 ug/ml | With |
| ple | Sex | Age | Tissue | ology | Type | Validation | Cell Type | IHC Score | IHC Score | IHC Score | Secondary |
| 1 | M | 70 | Bone | Normal | Normal | P | Erythroid precursor | 0 | 0 | 0 | 0 |
| | | | Marrow | | | | Myeloid precursor | 2 | 2-3 | 0-1 | 0 |
| | | | | | | | Megakaryocytes | 2-3 | 3 | 2-3 | 1 |
| | | | | | | | Macrophages | 1-2 | 2-3 | 2 | 0 |
| | | | | | | | Granulocytes | 3 | 3-4 | 0-1 | 0 |
| 2 | M | 58 | Bone | Normal | Normal | P | Erythroid precursor | 0 | 0 | 0 | 0 |
| | | | Marrow | | | | Myeloid precursor | 2 | 2 | 0 | 0 |
| | | | | | | | Megakaryocytes | 2-3 | 2-3 | 1 | 0 |
| | | | | | | | Macrophages | 1-2 | 2 | 0 | 0 |
| | | | | | | | Granulocytes | 3 | 3 | 0 | occ 2 |
| 3 | M | 76 | Bone | Normal | Normal | P | Erythroid precursor | 0 | 0 | 0 | 0 |
| | | | Marrow | | | | Myeloid precursor | 2 | 2 | 0-1 | 0 |
| | | | | | | | Megakaryocytes | 2-3 | 2-3 | 1 | 0 |
| | | | | | | | Macrophages | 2 | 2 | 1 | 0 |
| | | | | | | | Adipocytes | 2 | 2 | 2 | 2 |
| 4 | M | 16 | Liver | Normal | Normal | P | Hepatocytes | 0 | 0 | 0 | 0 |
| | | | | | | | Bile Duct Epithelium | 0-1 | 0 | 0 | 0 |
| | | | | | | | Kupffer Cells | 1-2 | 0-1 | 0 | 0 |
| | | | | | | | Other | 0 | 0 | 0 | 0 |
| 5 | M | 55 | Small | Normal | Normal | P | Epithelium | 0 | 0 | 0 | 0 |
| | | | Intestine | | | | Smooth Muscle | 0 | 0 | 0 | 0 |
| | | | | | | | Inflammatory | 0 | 0 | 0 | 0 |
| | | | | | | | Goblet Mucin, | 3 | 3 | 3 | 3 |
| | | | | | | | Brush B [symbol] | | | | |

[symbol]

indicates data missing or illegible when filed

Results With hOPG-hIgG1-Fc-FITC

hOPG-hIgG1-Fc-FITC, at a concentration of 5-10 μg/ml, showed faint to occasional moderate staining within lymphocytes in the tonsil, including the mantle zone, and within thymocytes in the thymus. Moderate staining was also seen in Hassall's corpuscles. The prostate was largely negative or showed occasional blush staining of smooth muscle. Within the testis, spermatogonia and occasional spermatocytes showed rare blush staining and Leydig cells were negative. The uterus showed blush staining of myometrial smooth muscle, and the ovary showed blush staining of stromal cells. The placenta showed moderate staining of syncytiotrophoblasts, and faint to moderate staining of endothelium and occasional stromal cells. The small intestine showed blush staining of epithelium with moderate to strong staining of the brush border and goblet cell mucin. Vessels, fibroblasts, ganglia, and smooth muscle of the submucosa and muscularis were negative. The liver section showed faint to moderate staining of hepatocytes, and faint to occasional moderate staining of sinusoidal lining cells.

Results with PROLIA-FITC:

PROLIA-FITC, at a concentration of 2.5-5 Vector g/ml, showed moderate to occasional strong staining within subsets of lymphocytes in the tonsil, particularly within the mantle zone of lymphoid follicles and faint to moderate staining of thymocytes, with occasional strong staining of medullary lymphocytes in the thymus. The prostate was negative for staining The testis showed rare blush staining of spermatogonia, but was largely negative in spermatocytes and Leydig cells. The uterus showed faint staining of myometrial smooth muscle, and the ovary section showed faint staining of vascular smooth muscle. The placenta showed moderate staining of syncytiotrophoblasts and faint to moderate staining of endothelium and occasional stromal cells. The small intestine showed faint staining of epithelium with moderate to strong staining of the brush border. Vessels, fibroblasts, ganglia, and smooth muscle of the submucosa and muscularis were negative. The liver section showed faint staining of hepatocytes.

Results with ENBREL-FITC:

ENBREL-FITC, at a concentration of 1.25 μg/ml, showed moderate to occasionally strong membranous staining of lymphocytes in the thymus and tonsil. The prostate showed faint to moderate staining of epithelial cells and faint staining of smooth muscle. The placenta showed moderate to occasionally strong staining of subsets of syncytiotrophoblasts faint to moderate staining of stromal cells and occasional cytotrophoblasts, and faint staining of endothelium. The ovary showed faint staining of stromal cells. The uterus sample showed faint staining of myometrial smooth muscle, and largely negative staining of vascular endothelium and vascular smooth muscle. Within the testis, spermatogonia and occasional spermatocytes showed moderate staining and Leydig cells were negative. The small intestine showed moderate staining of epithelium, strong staining of the brush border, and negative staining of submucosal vessels and fibroblasts.

In summary, hOPG-hIgG1-Fc-FITC at 5 μg/ml and PROLIA-FITC at 2.5 μg/ml showed positive staining within lymphocytes of the tonsil and thymus, with positive staining of sinusoidal endothelium of the splenic red pulp. Both reagents also showed positive staining of placental trophoblasts and endothelium, faint staining of uterine myometrial smooth muscle, and were largely negative in the prostate. Hepatocytes were also positive with both reagents. Cell types that showed positive staining were very similar between PROLIA-FITC and hOPG-hIgG1-Fc-FITC. ENBREL-FITC also showed positive staining of lymphocytes, placental trophoblasts and endothelium, and myometrial smooth muscle, but also showed positive staining in prostate epithelium and seminiferous tubules of the testis, in contrast to the other two reagents. The pattern of staining of ENBREL-FITC showed differences compared to PROLIA-FITC and hOPG-hIgG1-Fc-FITC, which were more similar to one another.

“Prolia”, “Xgeva” and “Enbrel” are registered trademarks of Amgen Inc., a Delaware Corporation.

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

What is claimed is:

1. A pharmaceutical composition inhibiting human RANKL, said composition comprising a polypeptide comprising:
a first amino acid sequence comprising amino acids 1 through 215 of human osteoprotegerin, and
a second amino acid sequence comprising amino acids 103 through 329 of human immunoglobulin gamma-1 Fc; and
wherein said polypeptide binds human RANKL with a Kd value of no more than about 5×10⁻¹³M.

2. The pharmaceutical composition of claim 1, wherein said polypeptide comprising amino acid sequence of SEQ ID NO. 1.

3. A therapeutic composition, the composition comprising a polypeptide that binds to human RANKL, said polypeptide comprising a biologically active portion of human osteoprotegerin and a Fc portion of human immunoglobulin gamma-1, wherein said polypeptide binds human RANKL with a Kd value of no more than about 5×10⁻¹³M.

4. The therapeutic composition of claim 3, further comprising about 25 mM sodium phosphate, from about 50 mM to about 100 mM NaCl, from about 20 to about 25 mM L-Arginine hydrochloride, and having pH value from about 6.3 to about 6.8.

5. The therapeutic composition of claim 4, further comprising about 10 mg/mL sucrose.

6. The therapeutic composition of claim 4, further comprising from about 10 mg/mL to about 25 mg/mL mannitol.

7. The therapeutic composition of claim 3, wherein half-life of said polypeptide in systemic circulation in Cynomolgus monkey after a subcutaneous administration at a dose of 3 mg/kg is at least 48 hours.

8. The therapeutic composition of claim 3, wherein half-life of said polypeptide in systemic circulation in Cynomolgus monkey after a subcutaneous administration at a dose of 10 mg/kg is at least 38 hours.

9. Use of a substance for manufacture of a medicament for the treatment or prevention of a disease associated with bone remodeling, the substance comprising a polypeptide comprising the amino acid sequence of SEQ ID NO. 1.

10. The use according to claim 9, wherein said disease is a carcinoma.

11. The use according to claim 9, wherein said disease is a breast cancer.

12. The use according to claim 9, wherein said disease is a prostate cancer.

13. The use according to claim 9, wherein said disease is multiple myeloma.

14. The use according to claim 9, wherein said disease is a bone sarcoma.

15. The use according to claim 9, wherein said disease is bone metastases due to solid tumors.

16. The use according to claim 9, wherein said disease is osteoporosis.

17. The use according to claim 9, wherein said disease is rheumatoid arthritis.

18. The use according to claim 9, wherein said disease is psoriatic arthritis.

19. A method of treating or preventing a disease or condition associated with bone remodeling, the method comprising administering to a patient in need for treating or preventing a disease associated with bone remodeling a therapeutically effective amount of a pharmaceutical composition comprising a polypeptide comprising the sequence of SEQ ID NO. 1.

20. The method of claim 19, wherein said disease is a metastatic carcinoma.

21. The use according to claim 19, wherein said disease is a carcinoma.

22. The use according to claim 19, wherein said disease is a breast cancer.

23. The use according to claim 19, wherein said disease is a prostate cancer.

24. The use according to claim 19, wherein said disease is multiple myeloma.

25. The use according to claim 19, wherein said disease is a bone sarcoma.

26. The use according to claim 19, wherein said disease is bone metastases due to solid tumors.

27. The use according to claim 19, wherein said disease is osteoporosis.

28. The use according to claim 19, wherein said disease is rheumatoid arthritis.

29. The use according to claim 19, wherein said disease is psoriatic arthritis.
